(19) United States
(12) Patent Application Publication
(10) Pub. No.: US 2022/0054659 A1
(43) Pub. Date: Feb. 24, 2022

(54) PSMA-TARGETED PAMAM DENDRIMERS FOR SPECIFIC DELIVERY OF IMAGING, CONTRAST AND THERAPEUTIC AGENTS

(71) Applicant: The Johns Hopkins University, Baltimore, MD (US)

(72) Inventors: Martin G. Pomper, Baltimore, MD (US); Wojciech G. Lesniak, Baltimore, MD (US); Srikanth Boinapally, Baltimore, MD (US); Sangeeta Banerjee Ray, Baltimore, MD (US); Catherine A. Foss, Baltimore, MD (US)

(21) Appl. No.: 17/312,548

(22) PCT Filed: Dec. 13, 2019
(86) PCT No.: PCT/US2019/066280

§ 371 Date: Jun. 10, 2021

Related U.S. Application Data
(60) Provisional application No. 62/779,884, filed on Dec. 14, 2018.

(51) Int. Cl. A61K 49/00; A61K 51/06

(52) U.S. Cl. CPC A61K 49/0054; A61K 51/065; A61K 49/0041; A61K 49/0002

(57) ABSTRACT

Prostate-specific membrane antigen (PSMA)-targeted PAMAM dendrimers (G4-PSMA) and their use for imaging or treating PSMA-expressing tumors or cells are disclosed.

### FEDERALLY SPONSORED RESEARCH OR DEVELOPMENT

This invention was made in part with United States Government support under CA134675, CA184228, CA183031, and EB024495 awarded by the National Institutes of Health (NIH). The government has certain rights in the invention.

### BACKGROUND

Prostate-specific membrane antigen (PSMA), also known as glutamate carboxypeptidase II (GCPII), or N-acetyl-L-aspartyl-L-glutamate peptidase I (NAALADase I), is a type II transmembrane glycoprotein responsible for the hydrolysis of N-acetylaspartyl glutamate (NAAG) to glutamate and N-acetylaspartate (NAA). PSMA is overexpressed in the epithelium of most prostate cancers (PC) compared to normal prostate tissue and benign hyperplasia and it has been associated with castration-resistant PC, metastasis, and poor prognosis. Ghosh and Heston, 2004; Chang et al., 2001; Wright et al., 1996; and Perner et al., 2007.

PSMA also is expressed on endothelial cells in the neovasculature of solid cancers other than PC, including lung, kidney, colon, stomach, breast, and brain cancers. Mhawech-Fauceglia et al. (2007); Wang et al., (2015); Haffner et al., 2009; Baccala et al., 2007; Fragomeni et al., 2017. The identification of PSMA substrate recognition sites has triggered extensive research leading to the development of numerous low-molecular-weight (LMW) PSMA inhibitors. Kiess et al., 2015; Maurer et al., 2016. These agents have been radiolabeled with several different radioisotopes and used for detection of PSMA expression in a variety of cancers with positron emission tomography (PET) and single photon emission computed tomography (SPECT). Kiess et al., 2015; Maurer et al., 2016. Among LMW PSMA inhibitors, the most widely studied are Lys-Glu-urea-based analogs, due to their facile synthesis, high PSMA binding affinity, specificity, and rapid internalization. Zhou et al., 2005; Kozikowski et al., 2001; Pomper et al., 2002.

Some LMW PSMA inhibitors are rapidly becoming important tools in the management of patients with prostate and other types of solid cancer, not only for detection and therapeutic monitoring, but also for endoradiotherapy. Fragomeni et al., 2017; Kiess et al., 2015; Mauer et al., 2016; Burger et al., 2017; Rowe et al., 2015; Sheikhbahaei et al., 2017; Delker et al., 2016; Rahbar et al., 2017. PSMA expression in solid cancers also has been successfully imaged with radiolabeled monoclonal antibodies, antigen-binding fragments (Fab2 and Fab′), and nanobodies in pre-clinical and clinical settings. Elgamal et al., 1998; Pandit-Taskar et al., 2015; Chatalic et al., 2015.

In addition to numerous compounds for nuclear imaging modalities, PSMA-specific agents for optical, magnetic resonance, photoacoustic and ultrasound imaging have been developed. Chen et al., 2009; Chen et al., 2017; Ray et al., 2017; Liu et al., 2017; Tavakoli et al., 2015; Wang et al., 2013. PSMA also has been utilized for the specific delivery of chemotherapeutics to solid tumors using antibody-drug conjugates (ADCs) and polylactic acid-polyethylene glycol (PLA-PEG)-based polymeric nanoparticles (BIND-014), which have undergone clinical evaluation. Petrylak et al., 2014; Galsky et al., 2008; Hrkach et al., 2012. Other PSMA-targeted nanoplatforms, such as aptamers, bionized nanoferrite (BNF), lipid-nanocarrier, polyethyleneimine-plasmid polyplex (pDNA-PEI), and iron oxide magnetic nanoparticles have been evaluated in pre-clinical studies. Farokhzad et al., 2006; Azad et al., 2015; Zhu et al., 2016; Bhatnagar et al., 2014; Tse et al., 2015.

This array of platforms demonstrates the versatility of the target in drug delivery, treatment with hyperthermia, endoradiotherapy, gene delivery, and as contrast material for magnetic resonance imaging, respectively. In early studies, Thomas and Patri et al., demonstrated PSMA-mediated in vitro uptake of generation-5 PAMAM dendrimers conjugated with fluorescein and J591 anti-PSMA monoclonal antibody by LNCaP cells.

Thomas et al., 2004; Patri et al., 2004. In a follow-up study, the same group showed specific in vitro toxicity for PAMAM dendrimer covalently modified with methotrexate and LMW PSMA inhibitor in LNCaP cells. Huang et al., 2014.

### SUMMARY

In some aspects, the presently disclosed subject matter provides a poly(amidoamine) (PAMAM) dendrimer comprising one or more prostate-specific membrane antigen (PSMA) targeting moieties, one or more optical imaging agents (IA), and one or more chelating moieties (Ch), wherein the one or more chelating moieties optionally comprise a metal or a radiometal suitable for radiotherapy and/or radioimaging, wherein the one or more prostate-specific membrane antigen (PSMA) targeting moieties, one or more optical imaging agents, and one or more chelating moieties are operably linked to the PAMAM dendrimer; or a pharmaceutically acceptable salt thereof.

In certain aspects, the PAMAM dendrimer is a compound of formula (I):

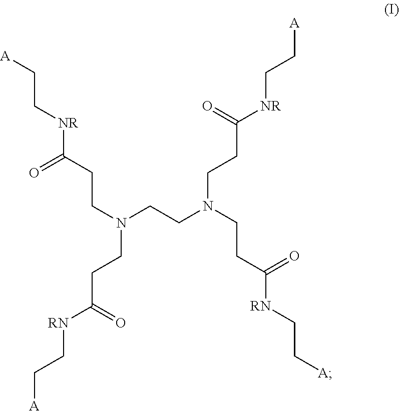

wherein each A is:

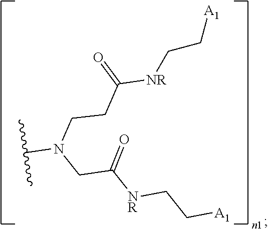

wherein each A₁ is selected from the group consisting of A, a prostate-specific membrane antigen (PSMA) targeting moiety, an optical imaging agent (IA), a chelating moiety (Ch), wherein the chelating moiety optionally comprises a metal or a radiometal suitable for radiotherapy and/or radioimaging, and an end-capping group (EC); n1 is an integer selected from the group consisting of 1, 2, 3, 4, 5, 6, 7, 8, 9, and 10; or a pharmaceutically acceptable salt thereof.

In particular aspects, the PAMAM dendrimer is a generation four (G4) PAMAM dendrimer. Other PAMAM dendrimers of generation 0, 1, 2, 3, 4, 5, 6, 7, 8, 9, and 10 also are suitable for use with the presently disclosed subject matter.

In certain aspects, the PSMA targeting moiety comprises a Lys-Glu-urea moiety having the following structure:

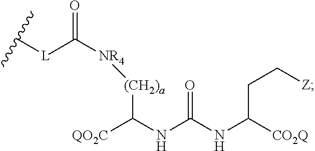

wherein: Z is tetrazole or CO₂Q; Q is H or a protecting group: a is an integer selected from the group consisting of 1, 2, 3, 4, and 5; R₄ is independently H, substituted or unsubstituted C₁-C₄ alkyl, or —CH₂—R₅; R₅ is selected from the group consisting of substituted or unsubstituted aryl and substituted or unsubstituted heteroaryl; and L is a linker. In particular embodiments, the linker is operably bound to the PAMAM dendrimer through a heterobifunctional crosslinker (CL).

In particular aspects, the optical imaging agent (IA) comprises a fluorescent dye. In yet more particular embodiments, the fluorescent dye is a near-infrared dye, including, but not limited to, rhodamine dye or derivative thereof. In particular aspects, the chelating moiety (CH) is 1,4,7,10-tetraazacyclododecane-1,4,7,10-tetraacetic acid (DOTA) or a DOTA analog, or any other metal chelator, such as diethylenetriamine pentaacetic acid (DTPA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO or deferoxamine), 1,4,7-triazacyclononane-N,N′,N″-triacetic acid (NOTA), and the like.

In some aspects, the PAMAM dendrimer further comprises a radiometal and the radiometal is selected from the group consisting of ⁶⁴Cu, ⁶⁷Cu, ⁶⁸Ga, ⁶⁰Ga, ⁸⁹Zr, ⁸⁶Y, ⁹⁰Y, ^{94m}Tc, ¹¹¹In, ⁶⁷Ga, ^{99m}Tc, ¹⁷⁷Lu, ⁵²Mn, ²¹³Bi, ²¹²Bi, ⁹⁰Y, ²¹¹At, ²²⁵Ac ²²³Ra, ^{186/188}Re, ¹⁵³Sm, Al¹⁸F, and ⁸⁹Sr.

In yet other aspects, the PAMAM dendrimer comprises an end-capping group (EC) selected from the group consisting of —NH₂, —(CH₂)ₘ₁—CH₂—CH(OR₁)—(CH₂)ₘ₁—OR₁, —NR—(CH₂)ₘ₁—CH(OR₁)—(CH₂)ₘ₁—OR₁, —NR—C(═O)—CH₃, —C(═O)—O⁻—Na⁺, —C(═O)—NR—(CH₂)ₘ₁—OR₁, —NR—C(═O)—(CH₂)ₘ₁—C(═O)OR₁, and —NR—(CH₂)ₘ₁—CH(OR₁)—(CH₂)ₘ₁—CH₃; wherein: each R is independently selected from the group consisting of H and C₁-C₄ alkyl; each R₁ is independently selected from the group consisting of H, Nat, C₁-C₄ alkyl, and a protecting group; and each m1 is independently an integer selected from the group consisting of 0, 1, 2, 3, 4, 5, 6, 7, 8, 9, 10, 11, and 12.

In other aspects, the PAMAM dendrimer further comprises a heterobifunctional crosslinker (CL). In certain aspects, the heterobifunctional crosslinker (CL) is succinimidyl 4-(N-maleimidomethyl)cyclohexane-1-carboxylate (SMCC).

In particular aspects, the PAMAM dendrimer has the following chemical structure:

wherein: m, n, p, q, and t are each independently integers from 0 to 64; Ch is a chelating moiety; CL is a heterobifunctional crosslinker; EC is an end-capping group; IA is an optical imaging agent; and PSMA is a PSMA-targeting moiety.

In yet more particular aspects, the PAMAM dendrimer has the following chemical structure:

In some aspects, the presently disclosed subject matter provides a pharmaceutical composition comprising a PSMA-targeted PAMAM dendrimer and a pharmaceutically acceptable carrier, diluent, or excipient.

In other aspects, the presently disclosed subject matter provides a method for imaging or treating one or more PSMA expressing tumors or cells, the method comprising contacting the one or more PSMA expressing tumors or cells with an effective amount of a PSMA-targeted PAMAM dendrimer, or a pharmaceutical composition thereof.

In particular aspects, the imaging or treating is in vitro, in vivo, or ex vivo.

In yet more particular aspects, the imaging is positron emission tomography (PET) and the radiometal is selected from the group consisting of 64Cu, ⁶⁷Cu, ⁶⁸Ga, ⁶⁰Ga, ⁸⁹Zr, ⁸⁶Y, and ^{94m}Tc.

In other aspects, the imaging is single-photon emission computed tomography (SPECT) and the radiometal is selected from the group consisting of ¹¹¹In, ⁶⁷Ga, ^{99m}Tc, and ¹⁷⁷Lu.

In yet other aspects, the treating comprises radiotherapy including a radiometal suitable for radiotherapy selected from the group consisting of ¹⁷⁷Lu, ²¹³Bi, ²¹²Bi, ⁹⁰Y ²¹¹At, ²²⁵Ac, ²²³R, and ⁸⁹Sr.

In certain aspects, the method comprises imaging or treating a cancer. In particular aspects, the cancer is selected from the group consisting of a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, FIG. 1D, FIG. 1E, and FIG. 1F show the purification and characterization of MP-Lys-Glu-urea and G4-PSMA. FIG. 1A and FIG. 1B are RP-HPLC and ESI-MS of MP-Lys-Glu-urea PSMA targeting moiety, demonstrating high purity of the PSMA-targeting moiety; FIG. 1C shows RP-HPLC purification of G4-PSMA, which was collected between 10 and 13 min of elution; FIG. 1D is an RP-HPLC profile of G4-PSMA with the UV-Vis spectrum recorded under the peak, indicating covalent attachment of rhodamine to the nanoparticles; FIG. 1E is MALDI-TOF spectra, illustrating an increase of the molecular weight upon each modification step of dendrimer terminal primary amines; and FIG. 1F is DLS of G4-PSMA, demonstrating narrow size distribution of the nanoparticles around 5 nm;

FIG. 2A, FIG. 2B, FIG. 2C, FIG. 2D, and FIG. 2E show the in vitro evaluation of G4-PSMA. FIG. 2A shows G4-PSMA binding to PSMA⁺ PC3 PIP and PSMA⁻ PC3 flu, approximately 1×10⁶ of each cell type was incubated with varied concentration of G4-PSMA, CI—confidence interval; FIG. 2B shows G4-PSMA binding to PSMA⁺ PC3-PIP in the absence and presence of 1 mM of ZJ-43, incubation was carried out using approximately 5×10⁵ cells; FIG. 2C shows competitive binding assay of G4-PSMA to PSMA⁺ PC3 PIP cells against ZJ-43, approximately 7×10⁶ cells were incubated with 1 μM of G4-PSMA and increasing concentration of ZJ-43 ranging from 1 μM to 1 mM; FIG. 2D is a summary of G4-PSMA in vitro binding to PSMA⁺ PC3-PIP and PSMA⁺ PC3-ful cell lines, **** P<0.001; and FIG. 2E is Epi-fluorescence microscopy of PSMA⁺ PC3 PIP, PSMA⁻ PC-3 flu cells after incubation with 150 nM of G4-PSMA or 150 nM of G4-PSMA plus 10 μM of ZJ-43 for 2 h at 37° C., scale bar: 50 μm. All panels show high in vitro PSMA specificity of G4-PSMA;

FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, FIG. 3M shows ex vivo biodistribution of G4-PSMA. Representative optical images of organs and tumors from mice bearing PSMA⁺ PC3 PIP and PSMA⁻ PC3 flu xenografts harvested 24 h post IV injection of: FIG. 3A—G4-PSMA (75 μg), FIG. 3B—G4-PSMA (75 μg) plus ZJ-43 (˜1 mg, n=3.29×10⁻⁶ mole) and FIG. 3C—saline. Images were acquired on a Xenogen IVIS Spectrum optical imaging system with excitation at 535 nm and emission at 580 nm; FIG. 3D is fluorescence images showing differential G4-PSMA uptake in PSMA⁺ PC3-PIP and PSMA⁻ PC3-flu tumors; FIG. 3E shows the semi-quantitative analysis of G4-PSMA accumulation in PSMA⁺ PC3 PIP and PSMA⁻ PC3 flu tumors, **** P<0.001; FIG. 3F, FIG. 3G, and FIG. 3H are epifluorescence microscopy illustrating distribution of G4-PSMA in PSMA⁺ PC3 PIP and PSMA⁻ PC3 flu xenografts and kidney acquired using freshly cut unstained sections; and FIG. 3I, FIG. 3J, FIG. 3K, FIG. 3L, FIG. 3M are epifluorescence microscopic images, illustrating PSMA expression and co-localization with G4-PSMA nanoparticles in PSMA⁺ PC3 PIP tumor as pointed by arrows;

FIG. 4A, FIG. 4B, and FIG. 4C show radiolabeling of G4-PSMA and in vitro evaluation of [⁶⁴Cu]G4-PSMA. FIG. 4A is a radio-HPLC chromatogram of the unpurified [⁶⁴Cu]G4-PSMA; FIG. 4B is a radio-HPCL profile of the [⁶⁴Cu]G4-PSMA obtained after ultrafiltration, showing high radiochemical purity of the radiotracer; and FIG. 4C shows in vitro binding of [⁶⁴Cu]G4-PSMA to PSMA⁺ PC3 PIP and PSMA− PC3 flu cell lines and blocking with 1 μM of ZJ-43 indicating PSMA specificity of nanoparticles, **** P<0.001;

FIG. 5 shows NOD-SCID mice bearing PSMA⁺ PC3 PIP and PSMA⁻ PC3 flu tumors in opposite flanks. One mouse was injected with approximately 200 μCi of [64Cu]G4-PSMA and imaged at 1 h, 24 h and 48 h post-injection;

FIG. 6A and FIG. 6B show in vivo evaluation of [⁶⁴Cu]G4-PSMA. FIG. 6A shows volume-rendered PET-CT images of NOD-SCID mice bearing PSMA⁺ PC3 PIP and PSMA⁻ PC3 flu xenografts injected with ˜ 9.25 MBq (250 μCi) of [⁶⁴Cu]G4-PSMA (upper panel) or ˜9.25 MBq (250 μCi) of [⁶⁴Cu]G4-PSMA with 50 mg/kg of ZJ-43 (lower panel); FIG. 6B shows ex vivo biodistribution of [⁶⁴Cu]G4-PSMA at 3 h, 24 h and 48 h after injection, in the same tumor model, ** P<0.02. Both PET-CT and biodistribution results indicate PSMA mediated [⁶⁴Cu]G4-PSMA uptake in PSMA⁺ PC3 PIP tumor;

FIG. 7A, FIG. 7B, and FIG. 7C show the synthesis and characterization of G4(Ctrl) control dendrimers. FIG. 7A is a schematic showing that generation 4 amine terminated PAMAM dendrimer was conjugated with on average two DOTA chelators and five molecules of rhodamine and remaining amines were capped with one hundred two butane-1,2-diol functionalities (the same G4(NH₂)₆₂(DOTA)₂ conjugate as for synthesis of G4-PSMA was used); FIG. 7B is MALDI-TOF spectra showing increase of the molecular weight upon each synthetic step; and FIG. 7C is DLS indicating narrow size distribution around 5 nm of G4(Ctrl) nanoparticles;

FIG. 8 shows ex vivo biodistribution of G4(Ctrl). Representative optical images of organs and tumors obtained from male NOD-SCID mice bearing PSMA⁺ PC3 PIP and PSMA⁻ PC3 flu xenografts harvested 24 h post IV injection of G4(Ctrl) and saline. Images were acquired on a Xenogen IVIS Spectrum optical imaging system with excitation at 535 nm and emission at 580 nm, scale was adjusted to the same minimum and maximum signal intensity as for G4-Ctrl analysis included in FIG. 3. Results indicate lack of preferential uptake of G4(Ctrl) in PSMA⁺ PC3 PIP vs. PSMA⁻ PC3 flu tumors and presence of nanoparticles in kidneys and bladder;

FIG. 9 shows ex vivo biodistribution of [⁶⁴Cu]G4-PSMA and [⁶⁴Cu]G4-Ctrl at 3 h, 24 h and 48 h after injection in male NOD-SCID mice bearing PSMA⁺ PC3 PIP and PSMA⁻ PC3 flu xenografts. Results indicate no preferential uptake of [⁶⁴Cu]G4(Ctrl) in PSMA⁺ PC3 PIP vs. PSMA⁻ PC3 flu and its fast renal clearance with minor hepatic accumulation; and

FIG. 10 is a representative generation four (G4) PAMAM dendrimer suitable for use with the presently disclosed subject matter (prior art).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. PSMA-Targeted PAMAM Dendrimers for Specific Delivery of Imaging, Contrast and Therapeutic Agents

A. PAMAM Dendrimers

The prostate-specific membrane antigen (PSMA) is a viable target for detecting and managing prostate cancer (PC). PSMA also is expressed in the neovasculature of many other solid cancers. Due to its fast internalization upon ligand binding, PSMA has been successfully utilized for endoradiotherapy and targeted drug delivery by antibody-drug conjugates and polymeric micelles (BIND-014).

Polyamidoamine (PAMAM) dendrimers are emerging as a versatile platform for drug delivery due to their unique physicochemical properties. The in vivo specificity, biodistribution, and clearance for PSMA-targeted dendrimers, however, have not yet been reported. The advantage of small PAMAM nanoparticles ranging in diameter from about 4 nm to about 6 nm compared to the relatively large antibody-drug conjugates (ADCs) or polymeric nanoparticles with size of about 50 nm to about 100 nm is their low off-target tissue uptake and preferential active tumor accumulation mediated by LMW targeting agents attached to dendrimers with less steric hindrances for binding to the target.

Accordingly, the presently disclosed subject matter provides generation four (G4) based PSMA-targeted PAMAM dendrimers (G4-PSMA) and evaluates their biological activity in vitro and in vivo using an experimental model of PC. In some embodiments, the Lys-Glu-urea low molecular weight PSMA inhibitor was used as a targeting moiety, as it has been reported to have suitable pharmacokinetics for in vivo targeting and imaging of PSMA.

The dendrimer also was conjugated with a fluorescent dye, in some embodiments, rhodamine, for optical imaging, and a chelating agent, in some embodiments, DOTA, for radiolabeling allowing nuclear imaging. The remaining terminal primary amines were capped with butane-1,2-diol. The presently disclosed G4-PSMA nanoparticles exhibited high in vitro target specificity and preferential accumulation in PSMA⁺ PC3 PIP xenografts vs. isogenic PSMA⁻ PC3 flu tumors, with predominant renal clearance and low off-target tissue uptake. Specific accumulation of G4-PSMA in PSMA⁺ PC3 PIP tumors was inhibited by the known PSMA inhibitor, ZJ-43. The presently disclosed subject matter demonstrates that G4-PSMA represents a suitable scaffold by which to target PSMA-expressing tissues with imaging/contrast, photodynamic therapy agents, silver and gold metallic nanoclusters, and therapeutics.

Accordingly, in some embodiments, the presently disclosed subject matter provides a poly(amidoamine) (PAMAM) dendrimer comprising one or more prostate-specific membrane antigen (PSMA) targeting moieties, one or more optical imaging agents (IA), and one or more chelating moieties (Ch), wherein the one or more chelating moieties optionally comprise a metal or a radiometal suitable for radiotherapy and/or radioimaging, wherein the one or more prostate-specific membrane antigen (PSMA) targeting moieties, one or more optical imaging agents, and one or more chelating moieties are operably linked to the PAMAM dendrimer; or a pharmaceutically acceptable salt thereof.

In some embodiments, the PAMAM dendrimer is a compound of formula (I):

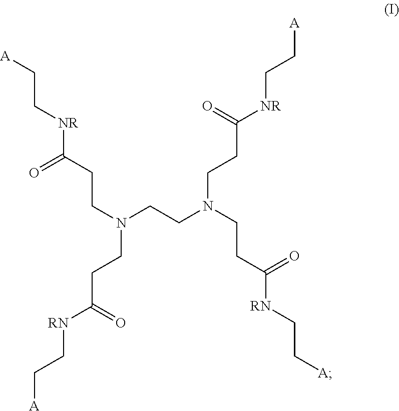

wherein: each A is:

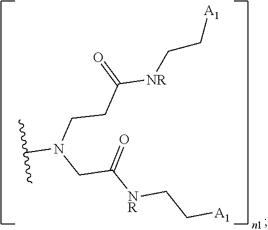

wherein each A₁ is selected from the group consisting of A, a prostate-specific membrane antigen (PSMA) targeting moiety, an optical imaging agent (IA), a chelating moiety (Ch), wherein the chelating moiety optionally comprises a metal or a radiometal suitable for radiotherapy and/or radioimaging, and an end-capping group (EC); n1 is an integer selected from the group consisting of 1, 2, 3, 4, 5, 6, 7, 8, 9, and 10; or a pharmaceutically acceptable salt thereof.

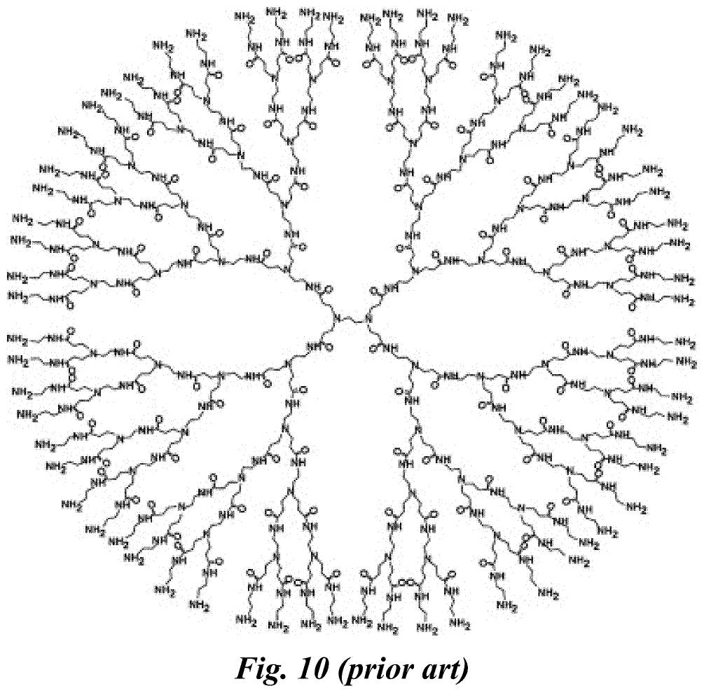

In particular embodiments, the PAMAM dendrimer is a generation four (G4) PAMAM dendrimer. Other PAMAM dendrimers of generation 0, 1, 2, 3, 4, 5, 6, 7, 8, 9, and 10 are suitable for use with the presently disclosed subject matter. A representative G4 PAMAM dendrimer suitable for use with the presently disclosed subject matter is provided in FIG. 10.

In certain embodiments, the PSMA targeting moiety comprises a Lys-Glu-urea moiety having the following structure:

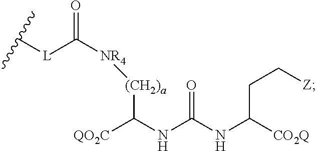

wherein: Z is tetrazole or CO₂Q; Q is H or a protecting group; a is an integer selected from the group consisting of 1, 2, 3, 4, and 5; R₄ is independently H, substituted or unsubstituted C₁-C₄ alkyl, or —CH₂—R₅; R₅ is selected from the group consisting of substituted or unsubstituted aryl and substituted or unsubstituted heteroaryl; and L is a linker.

In particular embodiments, the linker (L) is selected from the group consisting of —(CH₂)ₘ₁—, —C(═O)—(CH₂)ₘ₁—, —(CH₂—CH₂—O)ₜ₁—, —C(═O)—(CH₂—CH₂—O)ₜ₁—, —(O—CH₂—CH₂)ₜ₁—, —C(═O)—(O—CH₂—CH₂)ₜ₁—, —C(═O)—(CHR₂)ₘ₁—NR₃—C(═O)—(CH₂)ₘ₁—, —C(═O)—(CH₂)ₘ₁—O—C(═O)—NR₃—(CH₂)ₚ₁—, —C(═O)—(CH₂)ₘ₁—NR₃—C(═O)—O—CH₂)ₚ₁—, —C(═O)—(CH₂)ₘ—NR₃—C(═O)—NR₃—(CH₂)ₚ—, —C(═O)—(CH₂)ₘ—NR₃—C(═O)—(CH₂)ₚ₁—, —C(═O)—(CH₂)ₘ₁—C(═O)—NR₃—(CH₂)ₚ₁—, —C(═O)—(CH₂)ₘ₁—NR₃—C(═O)—NR₃—(CH₂)ₚ₁—, —C(═O)—(CH₂)ₘ₁—NR₁—C(═O)—NR₃—(CH₂)ₚ₁—, —C(═O)—(CH₂)ₘ₁—O—C(═O)—NR₃—, —C(═O)—CH₂)ₘ₁—O—C(═O)—NR₃—(CH₂)ₚ₁—, —C(═O)—(CH₂)ₘ₁—NR₃—C(═O)—O—(CH₂)ₚ₁—, polyethylene glycol, glutaric anhydride, albumin, and one or more amino acids; wherein each R is independently selected from the group consisting of H and C₁-C₄ alkyl; each R₁ is independently selected from the group consisting of H, Na⁺, C₁-C₄ alkyl, and a protecting group; each R₂ is independently selected from the group consisting of hydrogen, and —COOR₁; each R₃ is independently selected from the group consisting of hydrogen, substituted or unsubstituted linear or branched alkyl, alkoxyl, substituted or unsubstituted cycloalkyl, substituted or unsubstituted cycloheteroalkyl, substituted or unsubstituted aryl, substituted or unsubstituted heteroaryl, substituted or unsubstituted arylalkyl, and substituted or unsubstituted heteroarylalkyl; m1 and p1 are each independently an integer selected from the group consisting of 0, 1, 2, 3, 4, 5, 6, 7 and 8; t1 is an integer selected from the group consisting of 1, 2, 3, 4, 5, 6, 7, 8, 9, 10, 11 and 12; wherein the linker is operably bound to the PAMAM dendrimer through a heterobifunctional crosslinker (CL).

In certain embodiments, the optical imaging agent (IA) comprises a fluorescent dye. In particular embodiments, the fluorescent dye is selected from the group consisting of rhodamine, rhodamine B, rhodamine 6G, rhodamine 123, carboxytetramethylrhodamine (TAMRA), tetramethylrhodamine (TMR), tetramethylrhodamine-isothiocyanate (TRITC), sulforhodamine 101, Texas Red, Rhodamine Red, Rhodamine Green, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 635, AlexaFluor 647, BODIPY 630/650, BODIPY 650/665, BODIPY 581/591, BODIPY-FL, BODIPY-R6G, BODIPY-TR, BODIPY-TMR, BODIPY-TRX, Dy677, Dy676, Dy682, Dy752, Dy780, DyLight 350, DyLight 405, DyLight 488, DyLight 547, DyLight 550, DyLight 594, DyLight 633, DyLight 647, DyLight 650, DyLight 680, DyLight 755, DyLight 800, HiLyte Fluor 405, HiLyte Fluor 488, HiLyte Fluor 532, HiLyte Fluor 555, HyLyte Fluor 594, HiLyte Fluor 647, HiLyte Fluor 680, HiLyte Fluor 750, aminomethylcoumarin (AMCA), Cascade Blue, fluorescein, fluorescein isothiocyanate (FITC), Cy3, Cy5, Cy5.5, Cy7, 6-Carboxyfluorescein (6-FAM), and IRDye 800, IRDye 800CW, IRDye 800RS, IRDye 700DX, hexachlorofluorescein (HEX), 6-carboxy-4′,5′-dichloro-2′,7′-dimethoxyfluorescein (6-JOE), Oregon Green 488, Oregon Green 500, Oregon Green 514, Pacific Blue, REG, Renographin, ROX, TET, carbocyanine, indocarbocyanine, oxacarbocyanine, thuicarbocyanine, merocyanine, polymethine, coumarine, xanthene, a boron-dipyrromethane VivoTag-680, VivoTag-S680, VivoTag-S750, dimethyl{4-[1,5,5-tris(4-dimethylaminophenyl)-2,4-pentadienylidene]-2,5-cyclohexadien-1-ylidene}ammonium perchlorate (IR800), ADS780WS, ADS830WS, ADS832WS, R-Phycoerythrin, Flamma749, Flamma774, and indocyanine green (ICG), and N-hydroxysuccinimide (NHS) esters, maleimides, phosphines, and free acids thereof.

In some embodiments, the chelating moiety (CH) is 1,4,7,10-tetraazacyclododecane-1,4,7,10-tetraacetic acid (DOTA) or a DOTA analog, or any other metal chelator, such as diethylenetriamine pentaacetic acid (DTPA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO or deferoxamine), 1,4,7-triazacyclononane-N,N′,N″-triacetic acid (NOTA), and the like.

In particular embodiments, the chelating moiety (Ch) is selected from the group consisting of:

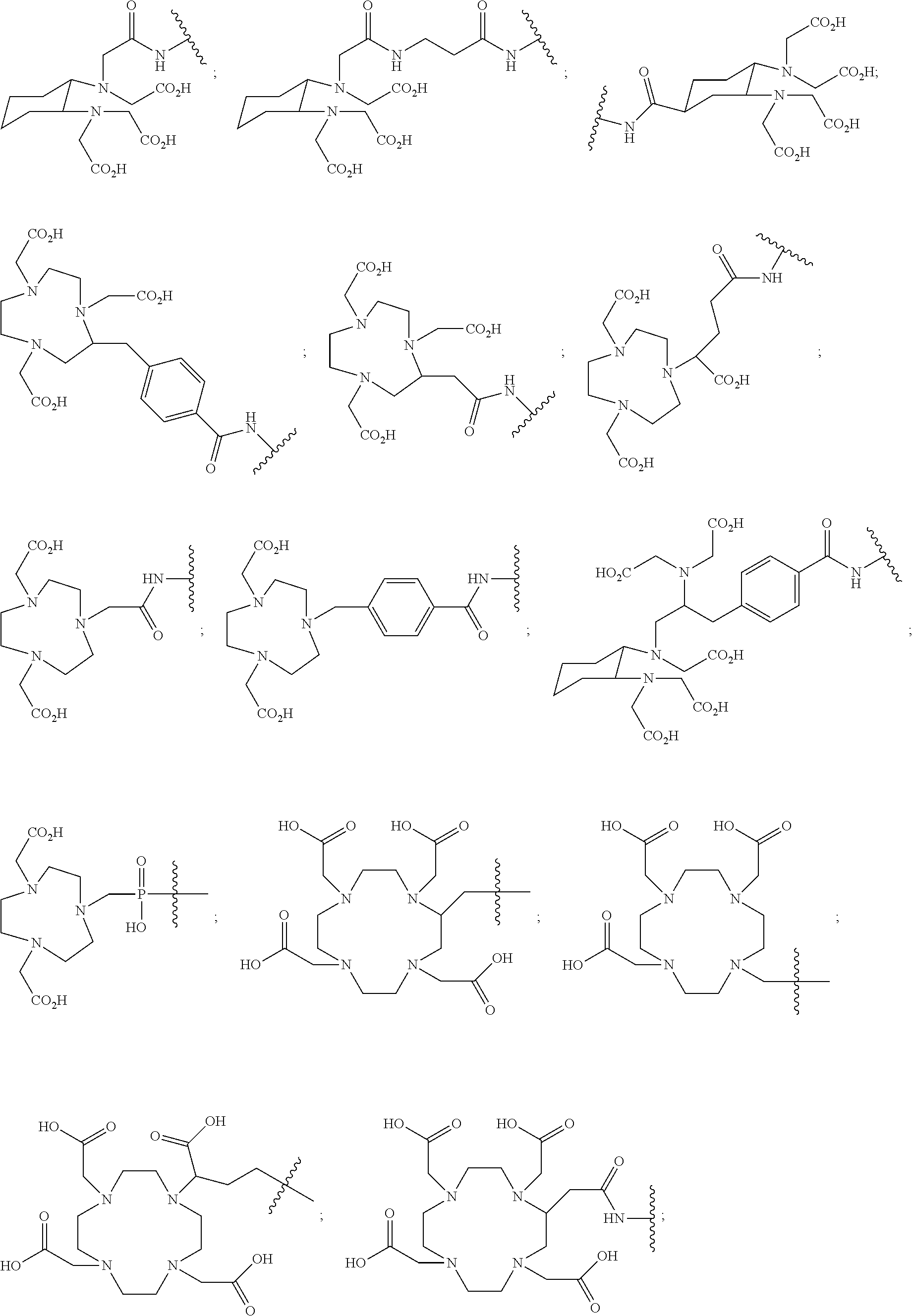

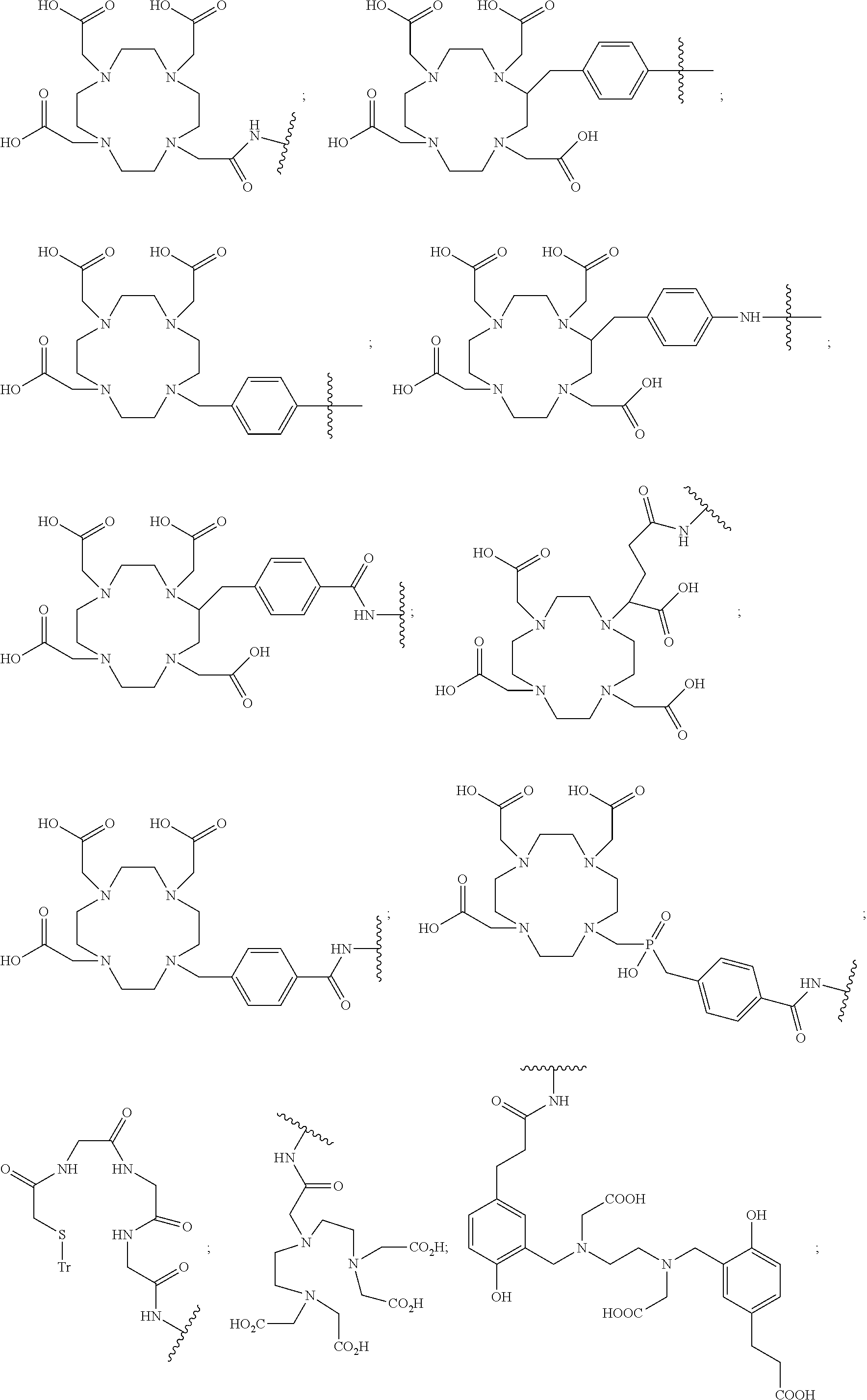

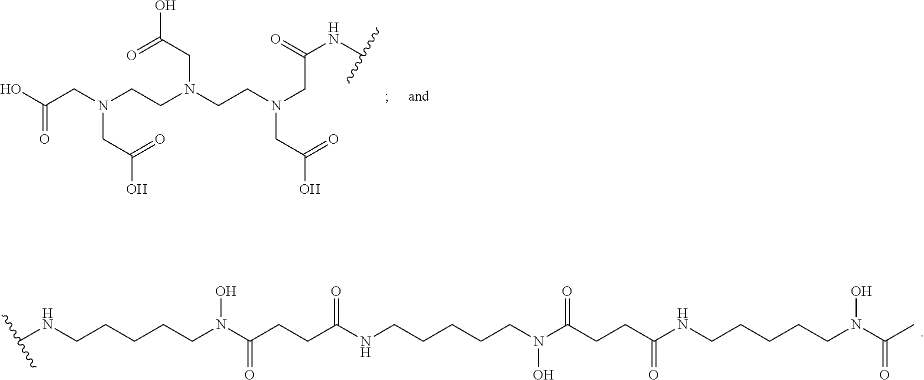 [SMILES: *NC(=O)CN(CCN(CCN(CC(=O)O)CC(=O)O)CC(=O)O)CC(=O)O ; *NCCCCCN(O)C(=O)CCC(=O)NCCCCCN(O)C(=O)CCC(=O)NCCCCCN(O)C(C)=O]

In particular embodiments, the chelating moiety is selected from the group consisting of:

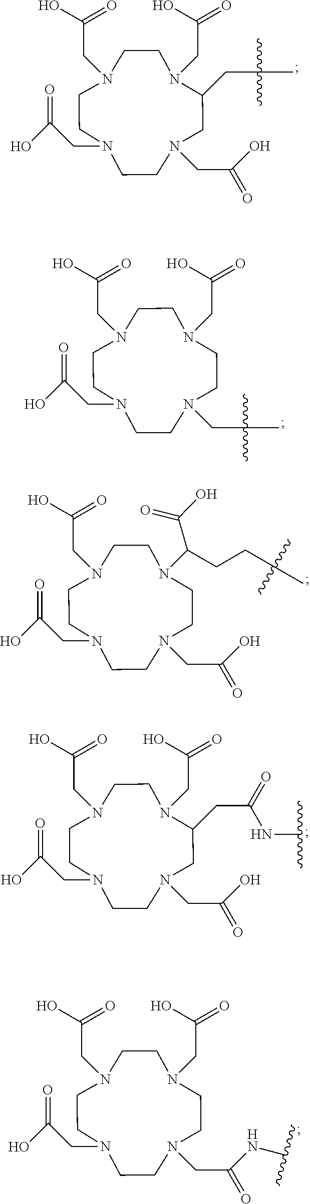 [SMILES: *CC1CN(CC(=O)O)CCN(CC(=O)O)CCN(CC(=O)O)CCN1CC(=O)O ; *CN1CCN(CC(=O)O)CCN(CC(=O)O)CCN(CC(=O)O)CC1 ; *CCC(C(=O)O)N1CCN(CC(=O)O)CCN(CC(=O)O)CCN(CC(=O)O)CC1 ; *NC(=O)CC1CN(CC(=O)O)CCN(CC(=O)O)CCN(CC(=O)O)CCN1CC(=O)O ; *NC(=O)CN1CCN(CC(=O)O)CCN(CC(=O)O)CCN(CC(=O)O)CC1]

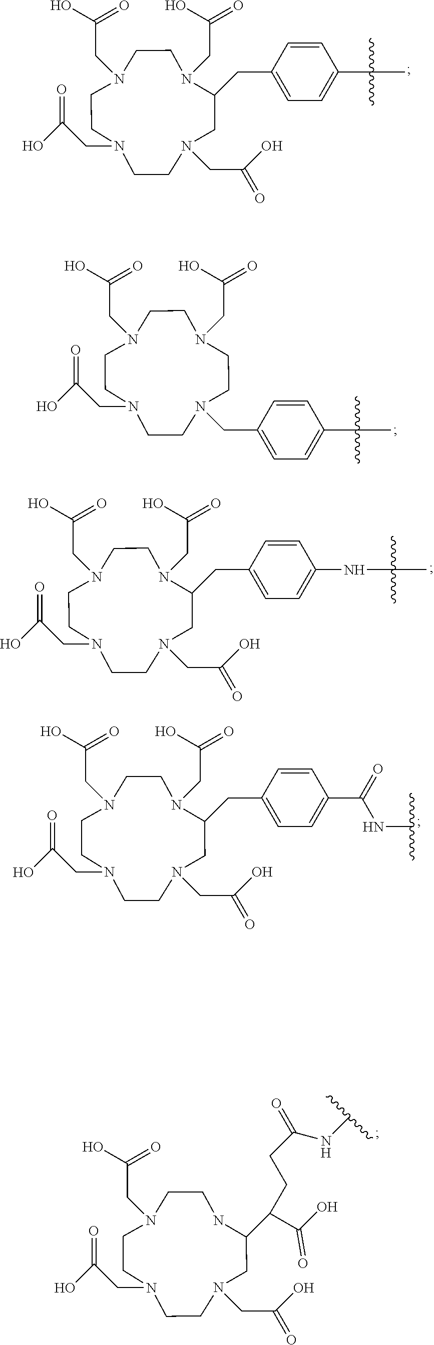

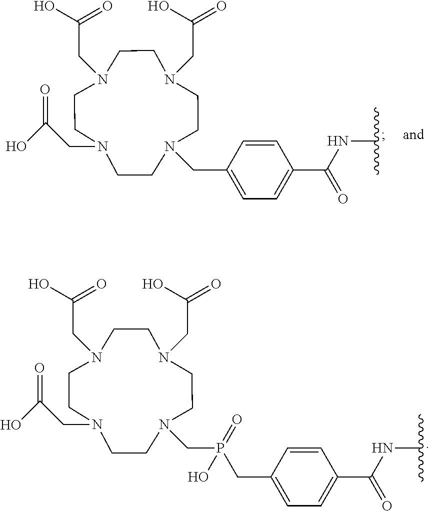 [SMILES: *NC(=O)c1ccc(CN2CCN(CC(=O)O)CCN(CC(=O)O)CCN(CC(=O)O)CC2)cc1 ; *NC(=O)c1ccc(CP(=O)(O)CN2CCN(CC(=O)O)CCN(CC(=O)O)CCN(CC(=O)O)CC2)cc1]

In some embodiments, the metal is selected from the group consisting of Cu, Ga, Zr, Y, Tc, In, Lu, Bi, Mn, Ac, Ra, Re, Sm, Al—F, and Sr. In particular embodiments, the metal is a radiometal and the radiometal is selected from the group consisting of ⁶⁴Cu, ⁶⁷Cu, ⁶⁸Ga, ⁶⁰Ga ⁸⁹Zr, ⁸⁶Y, ⁹⁰Y, ^{94m}Tc, ¹¹¹In, ⁶⁷Ga, ^{99m}Tc, ¹⁷⁷Lu, ⁵²Mn, ²¹³Bi, ²¹²Bi, ⁹⁰Y, ²¹¹At, ²²⁵Ac, ²²³Ra, ^{186/188}Re, ¹⁵³Sm, Al¹⁸F, and ⁸⁹Sr.

In some embodiments, the end-capping group (EC) is selected from the group consisting of —NH₂, —(CH₂)ₘ₁—CH₂—CH(OR₁)—(CH₂)ₘ₁—OR₁, —NR—(CH₂)ₘ₁—CH(OR₁)—(CH₂)ₘ₁—OR₁, —NR—C(═O)—CH₃, —C(═O)—O⁻Na⁺, —C(═O)—NR—(CH₂)ₘ₁—OR₁, —NR—C(═O)—(CH₂)ₘ₁—C(═O)OR₁, and —NR—(CH₂)ₘ₁—CH(OR₁)—(CH₂)ₘ₁—CH₃; wherein: each R is independently selected from the group consisting of H and C₁-C₄ alkyl; each R₁ is independently selected from the group consisting of H, Na⁺, C₁-C₄ alkyl, and a protecting group; and each m1 is independently an integer selected from the group consisting of 0, 1, 2, 3, 4, 5, 6, 7, 8, 9, 10, 11, and 12.

In some embodiments, the PAMAM dendrimer further comprises a heterobifunctional crosslinker (CL). In particular embodiments, the heterobifunctional crosslinker (CL) is selected from the group consisting of succinimidyl 4-(N-maleimidomethyl)cyclohexane-1-carboxylate (SMCC), m-maleimidobenzoyl-N-hydroxysuccinimide ester (MBS), N-(beta-maleimidopropyloxy)succinimide ester (BMPS), N-[e-maleimidocaproyloxy]succinimide ester (EMCS), N-[gamma-maleimidobutyryloxy] succinimide (GMBS), N-succinimidyl 4-[4-maleimidophenyl]butyrate (SMPB), succinimidyl-6-(β-maleimidopropionamido)hexanoate (SMPH), and maleimide-polyethylene glycol-N-hydroxysuccinimide ester (MAL-PEG-NHS).

In certain embodiments, the PAMAM dendrimer has the following chemical structure:

wherein: m, n, p, q, and t are each independently integers from 0 to 64; Ch is a chelating moiety; CL is a heterobifunctional crosslinker; EC is an end-capping group; IA is an optical imaging agent; and PSMA is a PSMA-targeting moiety.

In yet more certain embodiments, the PAMAM dendrimer has the following chemical structure:

B. Pharmaceutical Compositions and Administration

In another aspect, the present disclosure provides a pharmaceutical comprising a presently disclosed PSMA-targeted PAMAM dendrimer and a pharmaceutically acceptable carrier, diluent, or excipient. One of skill in the art will recognize that the pharmaceutical compositions include the pharmaceutically acceptable salts or hydrates of the compounds described above.

Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

C. Methods of Using the Presently Disclosed PAMAM Dendrimers or Pharmaceutical Compositions of Thereof

1. Method for Imaging or Treating PSMA Expressing Tumors or Cells

In some embodiments, the presently disclosed subject matter provides a method for imaging or treating one or more PSMA expressing tumors or cells, the method comprising contacting the one or more PSMA expressing tumors or cells with an effective amount of a presently disclosed PSMA-targeted PAMAM dendrimer, or a pharmaceutical composition thereof.

In certain embodiments, the imaging or treating is in vitro, in vivo, or ex vivo. In such embodiments, the method can be practiced by introducing, and preferably mixing, the compound and cell(s) or tumor(s) in a controlled environment, such as a culture dish or tube. The method can be practiced in vivo, in which case contacting means exposing the target in a subject to at least one compound of the presently disclosed subject matter, such as administering the compound to a subject via any suitable route. According to the presently disclosed subject matter, contacting may comprise introducing, exposing, and the like, the compound at a site distant to the cells to be contacted, and allowing the bodily functions of the subject, or natural (e.g., diffusion) or man-induced (e.g., swirling) movements of fluids to result in contact of the compound and the target.

In particular embodiments, the imaging is positron emission tomography (PET) and the radiometal is selected from the group consisting of ⁶⁴Cu, ⁶⁷Cu, ⁶⁸Ga, ⁶⁰Ga, ⁸⁹Zr, ⁸⁶Y, and ^{94m}Tc.

In other embodiments, the imaging is single-photon emission computed tomography (SPECT) and the radiometal is selected from the group consisting of ¹¹¹In, ⁶⁷Ga, ^{99m}Tc, and ¹⁷⁷Lu.

In yet other embodiments, the presently disclosed method further comprises diagnosing, based on the image, a disease or condition in a subject. In other embodiments, the presently disclosed method further comprises monitoring, based on the image, progression or regression of a disease or condition in a subject. In certain embodiments, the methods of the presently disclosed subject matter are useful for monitoring a site specific delivery of the therapeutic agent by localizing the dendrimer to the site in need of treatment and releasing the therapeutically active agent at the site in need of treatment.

In certain embodiments, the presently disclosed method for treating comprises radiotherapy. In yet more certain embodiments, the radiotherapy comprises a radiometal suitable for radiotherapy selected from the group consisting of ¹⁷⁷Lu, ²¹³Bi, ²¹²Bi, ⁹⁰Y ²¹¹At, ²²⁵Ac, ²²³R, and ⁸⁹Sr.

In particular embodiments, the presently disclosed method comprises imaging or treating a cancer. In yet more particular embodiments, the cancer is selected from the group consisting of a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

As used herein, the term “treating” can include reversing, alleviating, inhibiting the progression of, preventing or reducing the likelihood of the disease, or condition to which such term applies, or one or more symptoms or manifestations of such disease or condition.

“Preventing” refers to causing a disease, condition, or symptom or manifestation of such, or worsening of the severity of such, not to occur. Accordingly, the presently disclosed compounds can be administered prophylactically to prevent or reduce the incidence or recurrence of the disease, or condition.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

2. Uses of Presently Disclosed Dendrimers

In some embodiments, the presently disclosed dendrimers can be used as chelating agents, for example for forming gadolinium complexes suitable for use as magnetic resonance imaging (MRI) contrast agents; in photodynamic therapy, when conjugated with photosensitizers such porphyrins or Licor IRDye 700DX Dye, for example, see, “A PSMA-targeted theranostic agent for photodynamic therapy,” Chen Y, Chatterjee S, Lisok A, Minn I, Pullambhatla M, Wharram B, Wang Y, Jin J, Bhujwalla Z M, Nimmagadda S, Mease R C, and Pomper M G, J Photochem Photobiol B. 2017 167:111-116; in photoacoustic imaging, when conjugated with IRDye 800 WC Dye, for example, see “Prostate-specific membrane antigen-targeted photoacoustic imaging of prostate cancer in vivo,” Zhang H K, Chen Y, Kang J, Lisok A, Minn I, Pomper M G, Boctor E M, J Biophotonics. 2018 11(9):e201800021; and for drug delivery, for example, for delivering anti-cancer agents, including, but not limited to, maytansine, auristatin, methotrexate, and doxorubicin. In such embodiments, a dye, such as rhodamine, can be substituted with one or more drugs via one or more cleavable bonds. The presently disclosed dendrimers also can encapsulate drugs due to their large void volume. Representative uses of dendrimers for drug delivery are disclosed in “Nanoparticle Targeting of Anticancer Drug Improves Therapeutic Response in Animal Model of Human Epithelial Cancer,” Jolanta F. Kukowska-Latallo, Kimberly A. Candido,1 Zhengyi Cao, Shraddha S. Nigavekar, Istvan J. Majoros, Thommey P. Thomas, Lajos P. Balogh, Mohamed K. Khan, and James R. Baker, Jr., Cancer Res 2005; 65: (12). Jun. 15, 2005; “Potent Antitumor Activity of an Auristatin-Conjugated, Fully Human Monoclonal Antibody to Prostate-Specific Membrane Antigen,” Dangshe Ma, Christine E. Hopf, Andrew D. Malewicz, Gerald P. Donovan, Peter D. Senter, William F. Goeckeler, Paul J. Maddon, and William C. Olson, Clin Cancer Res 2006; 12(8) Apr. 15, 2006; “PEGylated PAMAM dendrimere doxorubicin conjugate-hybridized gold nanorod for combined photothermal-chemotherapy,” Xiaojie Li, Munenobu Takashima, Eiji Yuba, Atsushi Harada, and Kenji Kono, Biomaterials 2014 35, 6576-6584; and “Targeting HER2-Positive Breast Cancer with Trastuzumab-DM1, an Antibody-Cytotoxic Drug Conjugate,” Gail D. Lewis Phillips, Guangmin Li, Debra L. Dugger, Lisa M. Crocker, Kathryn L. Parsons, Elaine Mai, Walter A. Blattler, John M. Lambert, Ravi V. J. Chari, Robert J. Lutz, Wai Lee T. Wong, Frederic S. Jacobson, Hartmut Koeppen, Ralph H. Schwall, Sara R. Kenkare-Mitra, Susan D. Spencer, and Mark X. Sliwkowski, Cancer Res 2008; 68:9280-9290, each of which is incorporated herein in their entirety.

In other embodiments, the presently disclosed dendrimers can encapsulate metallic clusters, such as gold or silver metallic clusters, to form composite nanoparticles that can be used for photothermal therapy, computerized tomography (CT) imaging, and the like. See, e.g., “Enhanced optical breakdown in KB cells labeled with folate-targeted silver-dendrimer composite nanodevices,” Christine Tse, Marwa J. Zohdy, Jing Yong Ye, Matthew O'Donnell, Wojciech Lesniak, and Lajos Balogh, Nanomedicine: Nanotechnology, Biology and Medicine, 2011 7 (1), Issue 1, 97-106; and “Targeted CT/MR dual mode imaging of tumors using multifunctional dendrimer-entrapped gold nanoparticles,” Qian Chen, Kangan Li, Shihui Wen, Hui Liu, Chen Peng, Hongdong Cai, Mingwu Shen, Guixiang Zhang, and Xiangyang Shia, Biomaterials 2013 34(21), 5200-5209, each of which is incorporated by reference in its entirety.

In other embodiments, the presently disclosed dendrimers also can be used as nanodevices. See, e.g., “Synthesis and Characterization of PAMAM Dendrimer-Based Multifunctional Nanodevices for Targeting avP3 Integrins,” Wojciech G. Lesniak, Muhammed S. T. Kariapper, Bindu M. Nair, Wei Tan, Alan Hutson, Lajos P. Balogh, and Mohamed K. Khan, Bioconjugate Chemistry 2007 18 (4), 1148-1154, each of which is incorporated by reference in its entirety.

### II. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂— is equivalent to —OCH₂—; —C(═O)O—is equivalent to —OC(═O)—; —OC(═O)NR—is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic.

Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, alkylamino, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) 0, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′—and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

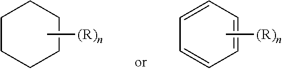

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

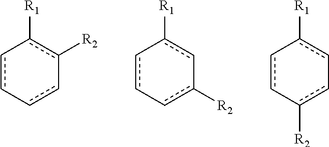

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R′R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

As used herein the term “monomer” refers to a molecule that can undergo polymerization, thereby contributing constitutional units to the essential structure of a macromolecule or polymer.

A “polymer” is a molecule of high relative molecule mass, the structure of which essentially comprises the multiple repetition of unit derived from molecules of low relative molecular mass, i.e., a monomer.

A “dendrimer” is highly branched, star-shaped macromolecules with nanometer-scale dimensions.

As used herein, an “oligomer” includes a few monomer units, for example, in contrast to a polymer that potentially can comprise an unlimited number of monomers. Dimers, trimers, and tetramers are non-limiting examples of oligomers.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)— catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

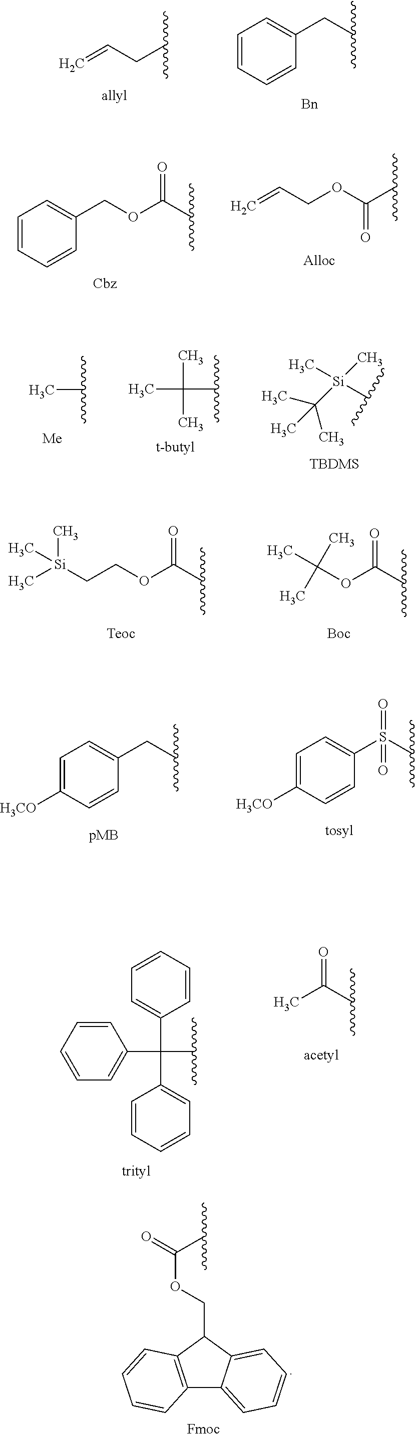

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### Study Design and Representative Results

The presently disclosed subject matter describes the synthesis of generation four (G4) PSMA-targeted PAMAM dendrimers (G4-PSMA) and their biological evaluation in vitro and in vivo using an experimental model of PC. A facile, one-pot synthesis gave nearly neutral nanoparticles with a narrow size distribution of approximately 5 nm in diameter and a molecular weight of 27,260 Da. G4-PSMA exhibited high in vitro target specificity with a dissociation constant (K_{d}) of 0.32±0.23 μM and preferential accumulation in PSMA⁺ PC3 PIP xenografts vs. isogenic PSMA⁻ PC3 flu tumors, with predominant renal clearance and low uptake in organs.

PET-CT and biodistribution studies of nanoparticles radiolabeled with copper-64, [⁶⁴Cu]G4-PSMA, demonstrated the highest PSMA⁺ PC3 PIP tumor accumulation at 24 h post-injection (45.83±20.09 percentage injected dose per gram of tissue, % ID/g) and PSMA⁺ PC3 PIP/PSMA⁻ PC3 flu ratio of 4.22±3.74, 7.65±3.35 and 3.94±1.09 at 3 h, 24 h and 48 h post-injection, respectively. Co-administration of [⁶⁴Cu]G4-PSMA with non-radiolabeled G4-PSMA nanoparticles resulted in decreased radioactivity retention in blood and all analyzed organs and tumors, leaving uptake ratios in PSMA⁺/PSMA⁻ tumors unaffected, indicating target specificity. Furthermore, specific accumulation of [⁶⁴Cu]G4-PSMA in PSMA⁺ PC3 PIP tumors was inhibited by the known PSMA inhibitor, ZJ-43. One advantage of G4-PSMA nanoparticles in targeted therapy, as compared to anti-PSMA antibody-drug conjugates or other relatively large polymeric nanoparticles with a size of between about 50 nm to about 100 nm, is their low off-target tissue accumulation, highly preferential uptake by PSMA positive tumors, and straightforward formulation.

### Example 2

### Synthesis of G4-PSMA

Synthesis of the PSMA-targeted (G4-PSMA) nanoparticles is presented in Scheme 1. To avoid off-target uptake (mainly liver and spleen uptake) and to achieve preferential renal clearance of PSMA-targeted dendrimers upon IV administration, generation-4 (G4) amine terminated PAMAM dendrimer with an approximately 4-nm hydrodynamic radius was selected as the starting material. A LMW Lys-Glu-urea inhibitor with picomolar affinity to PSMA was used.

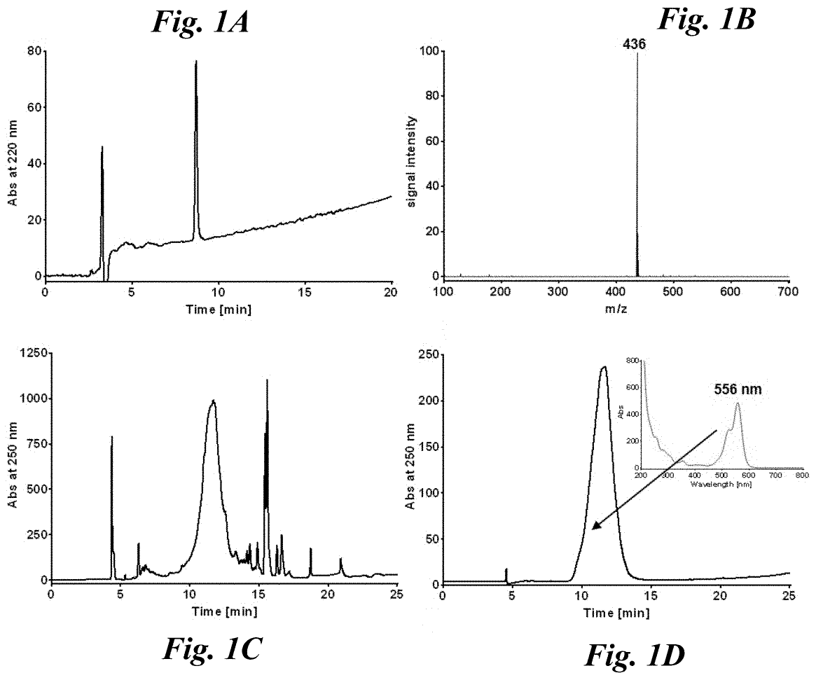

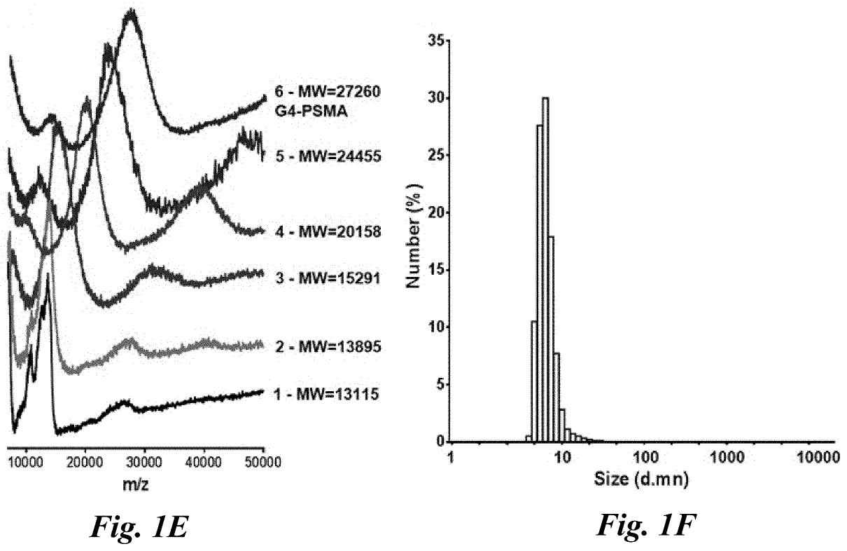

Prior conjugation with dendrimer Lys-Glu-urea was modified with 5-mercaptopentanoic acid to facilitate its conjugation to dendrimer via reaction with maleimide of the succinimidyl-4-[N-maleimidomethyl]cyclohexane-1-carboxylate (SMCC) heterobifunctional linker (Scheme 1). MP-Lys-Glu-urea was synthesized with high purity as demonstrated by RP-HPLC and ESI-Mass spectrometry (FIG. 1A and FIG. 1). G4-NH₂ was initially conjugated with two DOTA molecules (1), purified, lyophilized and used for further consecutive surface covalent attachment of three rhodamines (2), twenty-two SMCC linkers (3), of which ten reacted with MP-Lys-Glu-urea PSMA targeting moieties (4). In the final synthetic step, the remaining terminal amines were reacted with an excess of glycidol (5) to remove surface positive charge, which may lead to non-specific uptake and toxicity. Duncan and Izzo, 2005.

Reactions 2, 3, 4 and 5 were performed in a one-pot synthesis achieved by successive addition of reagents. G4-PSMA nanoparticles were initially purified using a PD10 column, followed by RP-HPLC purification (FIG. 1C), which yielded nanoparticles with a uniform RP-HPLC profile and UV-Vis spectrum, indicating covalent attachment of rhodamine (FIG. 1D). Prior to addition of SMCC, MP-Lys-Glu-urea, and glycidol, a small amount of reaction mixture was subjected to MADLI-TOF mass spectrometry, to confirm their covalent attachment to dendrimer. The average number of all conjugated moieties with dendrimer was derived from the consecutive increase of molecular weight upon each synthetic step as measured by MALDI-TOF (FIG. 1F). According to DLS analysis, the applied versatile synthetic route generated G4-PSMA nanoparticles of narrow size distribution with hydrodynamic radius of approximately 5 nm (FIG. 1F) and a zeta potential of −1.2 mV.

Referring now to Scheme 1A and Scheme 1B is (A) the synthesis of MP-Lys-Glu-urea PSMA targeting moiety and (B) a schematic of G4-NH₂ dendrimer surface modifications leading to formation of the presently disclosed PSMA-targeted nanoparticles. The number of conjugated functionalities was calculated based on the increase of molecular weight upon each synthetic step as detected by MALDI-TOF mass spectrometry.

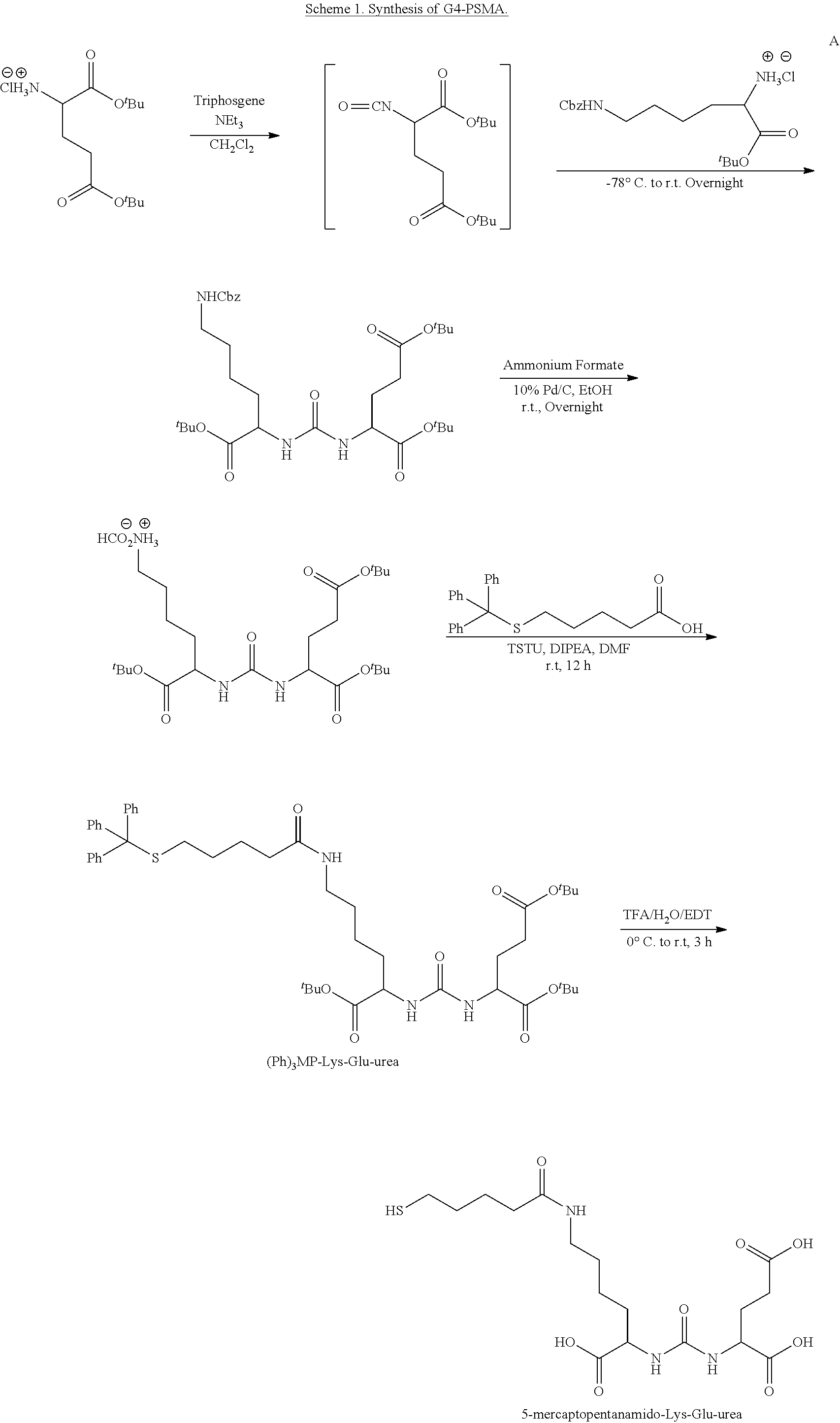

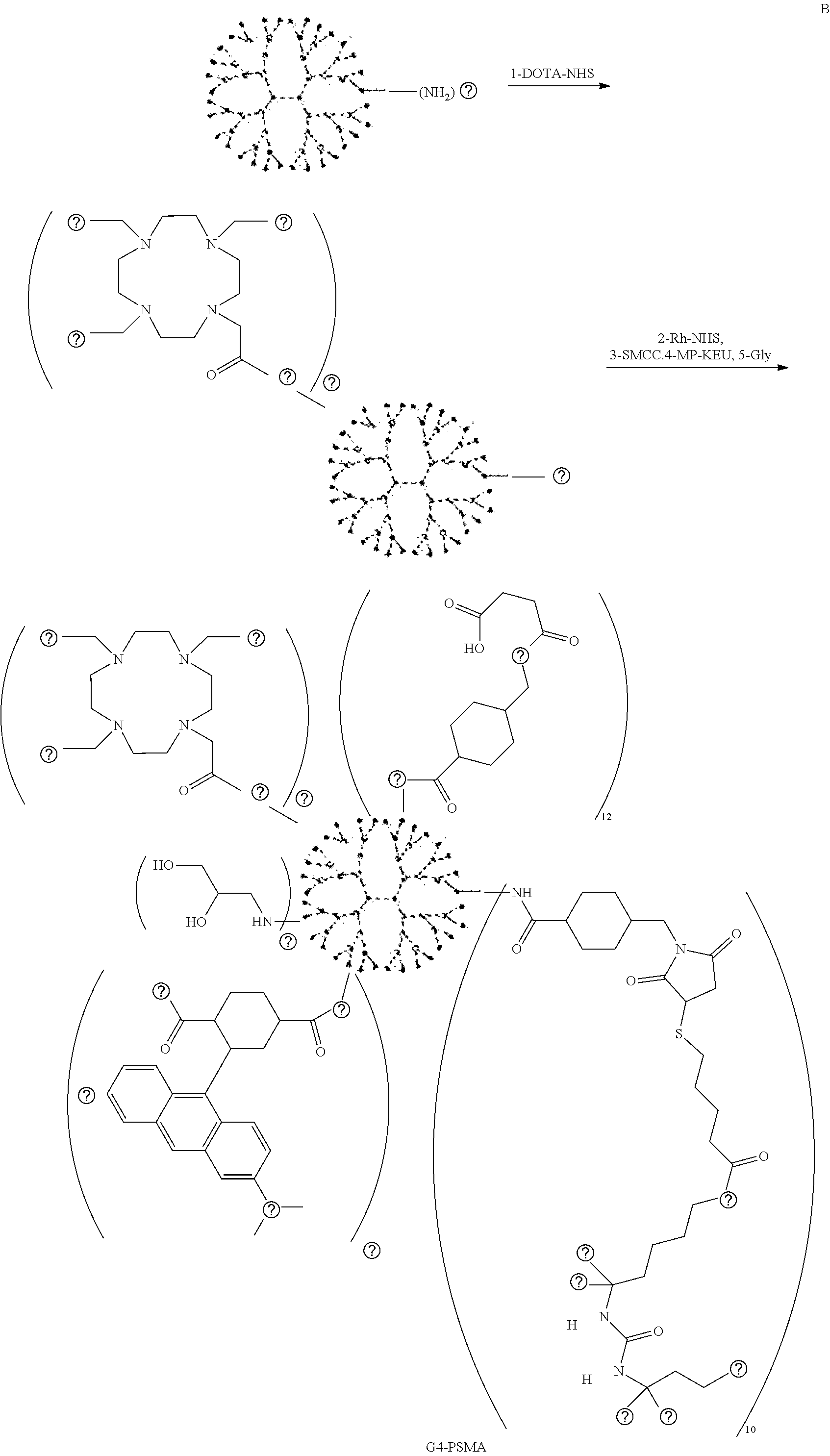

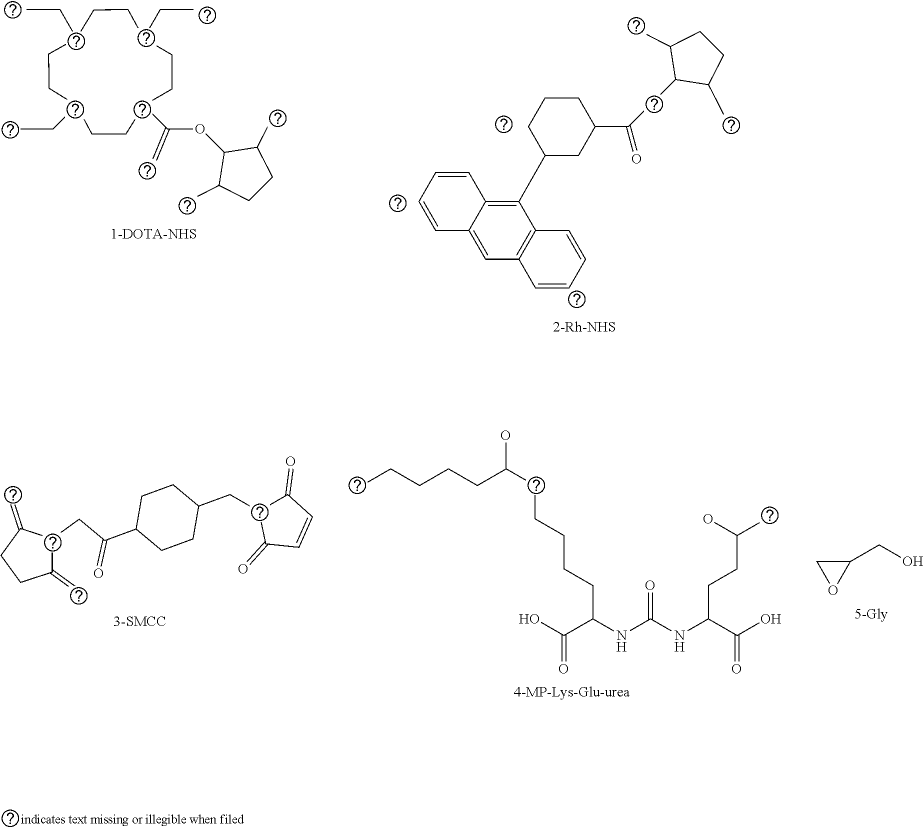 [SMILES: C=C(OC1C(C)CCC1C)C1CCC(CC)CCC(CC)CCC(CC)CC1 ; CC1CCC(C)C1CC(=O)C1CCCC(c2c3ccccc3cc3ccccc23)C1 ; C=C1CCC(=C)C1CC(=O)C1CCC(CC2C(=O)C=CC2=O)CC1 ; CCCCCC(O)CCCCCC(NC(=O)NC(CCC(C)O)C(=O)O)C(=O)O ; OCC1CO1]

Synthesis of MP-Lys-Glu-urea. The synthesis of thiol-terminated MP-Lys-Glu-urea commenced with the transformation of commercially available bromovaleric acid into 5-(tritylthio)pentanoic acid by treating with triphenyl methanethiol in the presence of sodium methoxide according to a previously reported protocol. Majer et al., 2003. This trityl derivative was converted to (Ph)₃MP-Lys-Glu-urea upon treatment with previously reported Glu-Lys-urea, Maresca et al., 2009, in the presence of N,N,N′,N′-tetramethyl-O—(N-succinimidyl)uronium tetrafluoroborate (TSTU) and DIPEA. Subsequent removal of trityl and tertiary butyl groups in the presence of TFA/H₂O/Ethanedithiol cocktail and purified by semi-preparative reverse phase high performance liquid chromatography (RP-HPLC) followed by lyophilization, which afforded MP-Lys-Glu-urea as a white solid in 42% overall yield.

5-(Tritylthio)pentanoic acid: To the oven dried round bottom flask, trityl mercaptan (150.8 mg, 0.545 mmol, 1.0 eq) was added under nitrogen atmosphere. The solid was dissolved in dry toluene (2 mL) with continuous stirring before the addition of a 30% (w/w) solution of sodium methoxide in methanol (220 μL, 1.2 mmol, 2.2 eq). To this mixture, a solution of bromovaleric acid (108.6 mg, 0.599 mmol, 1.1 eq) in methanol (1 mL) was slowly added at 5-10° C. The reaction mixture temperature was raised to 50° C. and then stirred for 2 h. The solvent was removed under reduced pressure and the residue was dissolved in 10 mL of water. The resulting aqueous solution was acidified (pH approximately 5-6) with 0.1 M H₂SO₄ and extracted with ethyl acetate (3×10 mL). The combined organic layers were dried over Na₂SO₄ and concentrated under reduced pressure. The crude product was recrystallized from EtOAc/hexanes to afford 5-(tritylthio)pentanoic acid as a white crystalline solid (142 mg, 70%). H¹-NMR (500 MHz, CDCl₃): δ 7.43-7.37 (m, 6H), 7.31-7.22 (m, 6H), 7.21-7.15 (m, 3H), 2.19 (t, J=2.2 Hz, 2H), 2.14 (t, J=2.1 Hz, 2H), 1.60-1.51 (m, 2H), 1.44-1.35 (m, 2H); C¹³—NMR (125 MHz, CDCl3): δ 179.8, 144.8, 129.6, 127.9, 126.6, 66.7, 33.5, 31.5, 28.1, 24.0.

Tri-tert-butyl(13S,17S)-7,15-dioxo-1,1,1-triphenyl-2-thia-8,14,16-triazanonadecane-13,17,19-tricarboxylate: 5-(tritylthio)pentanoic acid (120 mg, 0.319 mmol, 1.0 eq) and N,N,N′,N′-Tetramethyl-O—(N-succinimidyl)uronium tetrafluoroborate (TSTU) (96 mg, 0.319 mmol, 1.0 eq) were dissolved in DMF (2 mL). To the above mixture was added Glu-Lys-urea (187 mg, 0.351 mmol, 1.1 eq) and diisopropylethylamine (144 mg, 1.11 mmol, 3.5 eq) dissolved in DMF (2 mL) dropwise for 10 min. The resulted solution was stirred overnight at room temperature. The solvent was removed under reduced pressure and purified through silica gel chromatography using EtOAc/hexanes (50% EtOAc in hexanes) to afford compound 3 as a white solid (215 mg, 80%). H¹-NMR (500 MHz, CDCl₃): δ 7.39 (d, J=7.7 Hz, 5H), 7.32-7.17 (m, 8H), 7.20 (t, J=7.4 Hz, 3H), 6.15-5.95 (m, 1H), 5.50-5.15 (m, 2H), 4.35-4.20 (m, 2H), 3.30-3.07 (m, 2H), 2.40-2.22 (m, 2H), 2.14 (t, J=7.2 Hz, 2H), 2.10-2.02 (m, 3H), 1.90-1.68 (m, 2H), 1.63-1.53 (m, 3H), 1.45 (s, 9H), 1.44 (s, 9H), 1.43 (s, 9H), 1.52-1.27 (m, 6H); C¹³—NMR (125 MHz, CDCl₃): δ 173.2, 173.1, 172.4, 172.2, 157.3, 145.0, 129.6, 127.9, 126.6, 82.4, 81.6, 80.6, 66.4, 53.5, 53.1, 39.1, 36.1, 32.5, 31.7, 29.0, 28.3, 28.1, 25.3, 22.9; MS (ESI): m/z 868.4 (M+Na).

(S)-1-Carboxy-5-(5-mercaptopentanamido)pentyl)carbamoyl)-L-glutamicacid (MP-Lys-Glu-urea): 5 mL mixture of TFA/H₂O/Ethanedithiol (94:3:3) was added to the round bottom flask containing compound 3 (100 mg, 0.118 mmol) at 0° C. The reaction mixture was stirred for 3 h at room temperature and concentrated under reduced pressure. The crude was purified by preparative RP-HPLC chromatography using 0.1% TFA in H₂O and 0.1% TFA in acetonitrile as eluents followed by lyophilization to afford compound 4 as a white solid (38.5 mg, 75%). RP-HPLC purification was achieved using Agilent System, k 220 nm, 250 mmx 10 mm Phenomenex Luna C18 column, solvent gradient: 90% H₂O (0.1% TFA) and 10% ACN (0.1% TFA), reaching 60% of ACN in 20 min at a flow rate of 5 mL/min, product eluted at 8.7 min. H¹-NMR (400 MHz, CDCl₃): δ 7.75 (t, J=5.7 Hz, 1H), 6.30 (dd, J=8.4, 13.0 Hz, 2H), 4.12-3.99 (m, 2H), 2.99 (q, J=6.2 Hz, 2H), 2.52-2.47 (m, 1H), 2.44 (t, J=6.6 Hz, 2H), 2.22 (t, J=7.4 Hz, 2H), 2.03 (t, J=7.1 Hz, 2H), 1.97-1.85 (m, 1H), 1.77-1.19 (m, 1OH); MS (ESI): m/z 436.1 (M+H).

Synthesis of G4-PSMA nanoparticles. Preparation of G4-PSMA involved a multi-step synthesis as presented in Scheme 1B. (step 1) 0.229 g (1.61×10⁻⁵ mole) of G4-NH₂ dendrimer was dissolved 10 mL 1×PBS buffer, placed in round bottom flask and 2 mole equivalent of DOTA-NHS-ester (0.0245 g, 3.22×10⁻⁵ mole) reconstituted in 0.2 mL of DMSO was added. Reaction was carried for 2 h at room temperature (RT), followed by dialysis against deionized water using a regenerated cellulose membrane with 10,000 Da molecular weight cut-off (MWCO). Then excess water was evaporated and the residue lyophilized, which provided 0.249 g of the GNH₂-DOTA conjugate. Conjugation of rhodamine, MP-Lys-Glu-urea and capping of primary amine with glycidol was achieved in one-pot synthesis. (step 2) 0.0219 g (1.46×10⁻⁶ mole) of GNH₂-DOTA conjugate was dissolved in 5 mL of 1×PBS and mixed with 0.1 mL of DMSO containing 0.0038 g (5.48×10⁻⁶ mole) of rhodamine-NHS ester. After 2 h of stirring at RT a small amount of reaction mixture was subjected to MALDI-TOF mass spectrometry (as describe below) to confirm conjugation of rhodamine with GNH₂-DOTA. Next, 0.0078 g (2.33×10⁻⁵ mole) succinimidyl-4-[N-maleimidomethyl]cyclohexane-1-carboxylate (SMCC) heterobifunctional linker dissolved in 0.1 mL of DMSO was added and reacted for 1 h (step 3), followed by MADLI-TOF mass spectrometry analysis to confirm covalent attachment of SMCC linker with G4-NH₂-DOTA-rhodamine conjugate. Subsequently, 0.0127 g (2.99×10⁻⁵ mole) of MP-Lys-Glu-urea dissolved in 0.5 mL of 1×PBS was added into reaction mixture and allowed to react for 1 h (step 4), followed by MADLI-TOF mass spectrometry analysis to confirm covalent attachment of MP-Lys-Glu-urea with G4-NH₂-DOTA-rhodamine-MCC maleimide activated nanoparticles. Then 0.1 mL of 4M NaOH and 0.2 mL (2.99×10⁻³ mole) of glycidol was added and reaction was carried for additional 16 h to cap remaining unmodified primary amines with butane-1,2-diol (step 5) and provide PSMA-targeted nanoparticles (G4-PMSA). G4-PSMA was initially purified using PD10 size exclusion column (GE Healthcare), followed by purification on a RR-HPLC system (Varian ProStar) equipped with an Agilent Technology 1260 Infinity photodiode array detector using a semi-preparative C-18 Luna column (5 mm, 10×25 mm Phenomenex) and a gradient elution starting with 98% H₂O (0.1% TFA) and 2% ACN (0.1% TFA), reaching 100% of ACN in 30 min at a flow rate of 4 mL/min. G4-PSMA was collected between 10 and 13 min of elution. This fraction was evaporated using rotary evaporator, and the obtained residue was dissolved in deionized water and lyophilized, yielding 0.031 g of red powder.

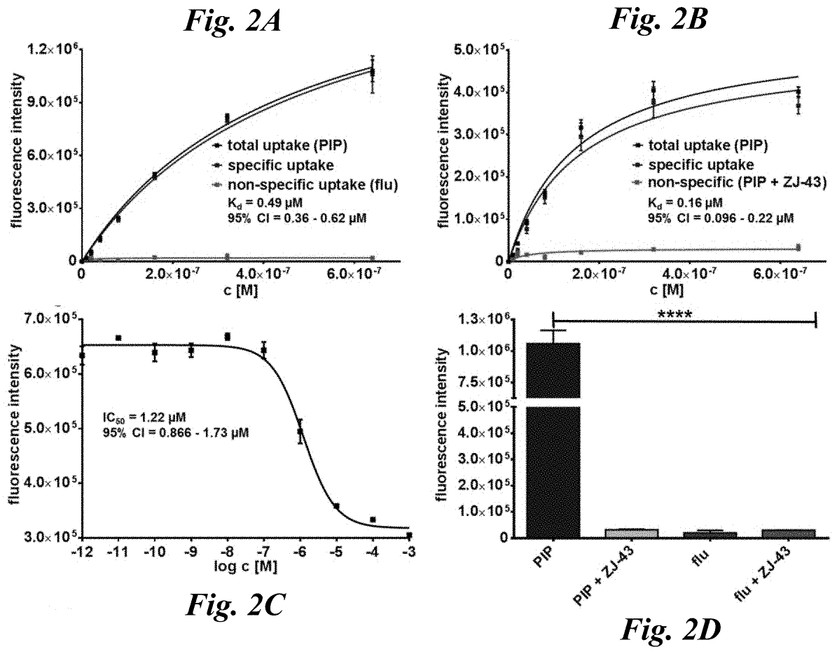

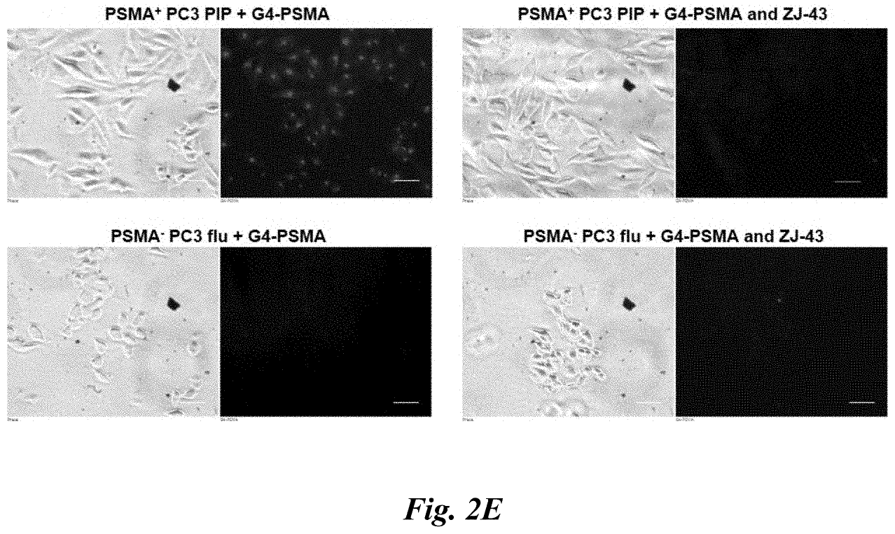

In vitro evaluation of G4-PSMA specificity. To assess G4-PSMA specificity in vitro and affinity to PSMA, several different assays were carried out using isogenic human prostate cancer PSMA⁺ PC3 PIP and PSMA⁻ PC3 flu cell lines (FIG. 2). As presented in FIG. 2A, a concentration-dependent increase of fluorescence intensity upon addition of G4-PSMA was observed in PSMA⁺ PC3 PIP cells. In contrast, in PSMA⁻ PC3 flu cells the signal intensity remained unchanged in the entire G4-PSMA concentration range applied, suggesting highly specific G4-PSMA nanoparticles binding to PSMA with a derived K_{d} value of 0.49 μM (95% confidence interval 0.36-0.62 μM, Bₘₐₓ=1.91×10⁶). Pre-mixing of PSMA⁺ PC3 PIP cells with 1 mM of ZJ-43 resulted in complete inhibition of G4-PSMA uptake (FIG. 2B) that provided a K_{d} value of 0.16 μM (95% confidence interval 0.10-0.22 μM, Bₘₐₓ=5.05×10⁵). Next a competitive binding assay was carried out using PSMA⁺ PC3 PIP cells and 1 μM of G4-PSMA against varied concentration of ZJ-43 (FIG. 2C). The assay provided an IC₅₀ value of 1.22 μM (95% confidence interval 0.87-1.73 μM), indicating that approximately 10-fold higher concentration of ZJ-43 is required to inhibit interaction of G4-PSMA with PSMA⁺ PC3 PIP cells.

In vitro cellular uptake of G4-PSMA by PSMA⁺ PC3 PIP and PSMA⁻ PC3 flu cells was also evaluated by epifluorescence microscopy (FIG. 2E). Internalization of G4-PSMA was observed in PSMA⁺ PC3 PIP following 2 h of incubation at 37° C., which could be inhibited by excess ZJ-43. In contrast there was no detectable internalization of the G4-PSMA nanoparticles in PSMA⁻ PC3 flu cells.

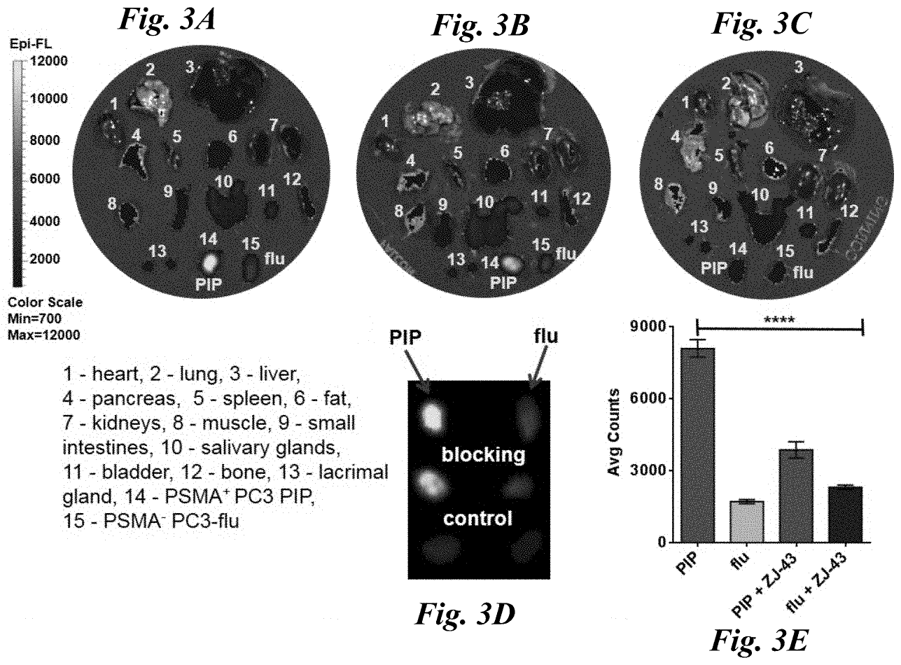

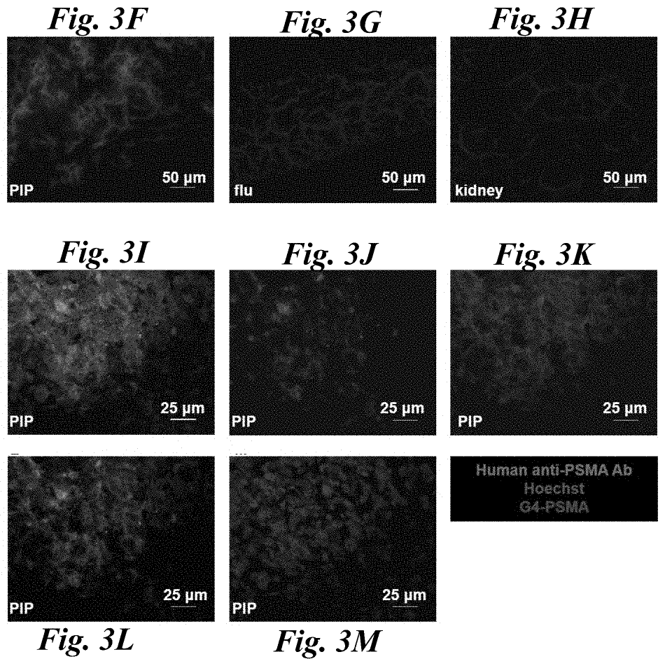

Optical imaging. Prompted by the promising in vitro results an ex vivo evaluation of G4-PSMA in NOD-SCID mice bearing subcutaneous PSMA⁺ PC3 PIP and PSMA⁻ PC3 flu xenografts in opposite flanks was undertaken with optical imaging. The upper panel of FIG. 3 illustrates representative images of tissues dissected from mice 24 h after IV injection of G4-PSMA (FIG. 3A), G4-PSMA plus ZJ-43 (FIG. 3B), and saline (FIG. 3C).

High fluorescence intensity could be detected in PSMA⁺ PC3 PIP tumors. Only marginally increased signal intensity compared to background was detected in PSMA⁻ PC3 flu tumors, salivary glands, kidneys, pancreas, liver and bladder, indicating specific G4-PSMA accumulation in PSMA-expressing tumors. Semi-quantitative analysis of G4-PSMA accumulation in tumors provided a PSMA⁺ PC3 PIP/PSMA⁻ PC3 flu ratio of 4.76±0.02 (FIG. 2D, FIG. 2E). Co-administration of G4-PSMA with ZJ-43 resulted in decreased nanoparticle uptake in PSMA⁺ PC3 PIP tumors by more than 50% and complete clearance from kidneys, confirming PSMA-mediated uptake of G4-PSMA. Sections obtained from imaged PSMA⁺ PC3 PIP tumors, PSMA⁻ PC3 flu tumors and kidneys were further analyzed with epifluorescence microscopy (FIGS. 2F-2M). In agreement with whole tumor and organ images, higher accumulation of G4-PSMA within PSMA⁺ PC3 PIP tumors in comparison to PSMA⁻ PC3 flu tumors and kidneys was detected in freshly cut, unstained sections (FIG. 3F, FIG. 3G, and FIG. 3H). After staining of PSMA and cell nuclei, fluorescence related to G4-PSMA remained in samples obtained from PSMA⁺ PC3 PIP tumors (FIGS. 3I-3M). The co-localization of PSMA expression and G4-PSMA distribution further verified PSMA-mediated uptake of the nanoparticles. In contrast, the same procedure resulted in failure to detect G4-PSMA nanoparticles in samples acquired from PSMA⁻ PC3 flu tumor and kidneys.

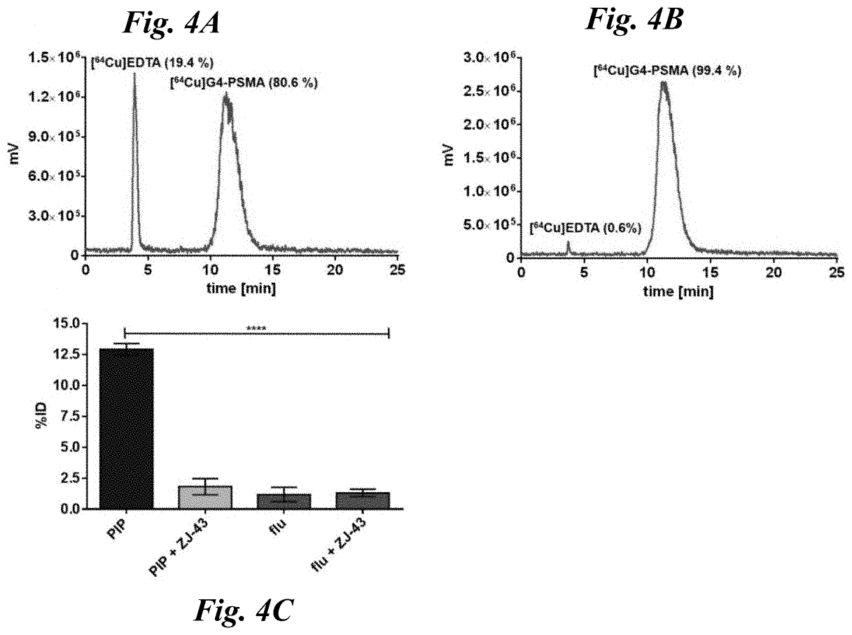

Radiolabeling and in vitro evaluation of [⁶⁴C]G4-PSMA specificity. The radiolabeling of G4-PSMA with ⁶⁴Cu was carried out for 30 min in acetate buffer at pH approximately 4.5 and at 85° C. Subsequently, EDTA was added into the reaction mixture to a final concentration of 5 mM, and incubation was continued for additional 5 min to chelate free or loosely bound [⁶⁴Cu]. Radio-HPLC chromatogram of the reaction mixture (FIG. 4A) showed an 80.6% G4-PSMA radiolabeling efficiency. Next, [⁶⁴Cu]G4-PSMA was purified via centrifugal ultrafiltration, which yielded radiotracer with a high specific activity of 70.67 MBq/μmol (1.91 Ci/μmol) and 99.4% radiochemical purity (FIG. 4C). For further studies [⁶⁴Cu]G4-PSMA was diluted with saline.

To assess PSMA binding properties of [⁶⁴Cu]G4-PSMA, in vitro binding assays were performed in PSMA⁺ PC3 PIP and PSMS⁻ PC3 flu cell lines (FIG. 4C). [⁶⁴Cu]G4-PSMA demonstrated higher uptake in PSMA⁺ PC3 PIP cells (12.92±0.47 percent incubated dose, % ID), compared to PSMA⁻ PC3 flu (1.18±0.58% ID). The specific uptake of [⁶⁴Cu]G4-PSMA by PSMA⁺ PC3 PIP cells could be blocked with 1 mM of ZJ-43, further confirming the target specificity of radiolabeled nanoparticles. In contrast, ZJ-43 did not influence uptake of [⁶⁴Cu]G4-PSMA in PSMA⁻ PC3 flu cells, which remained at 1.05±0.25% ID.

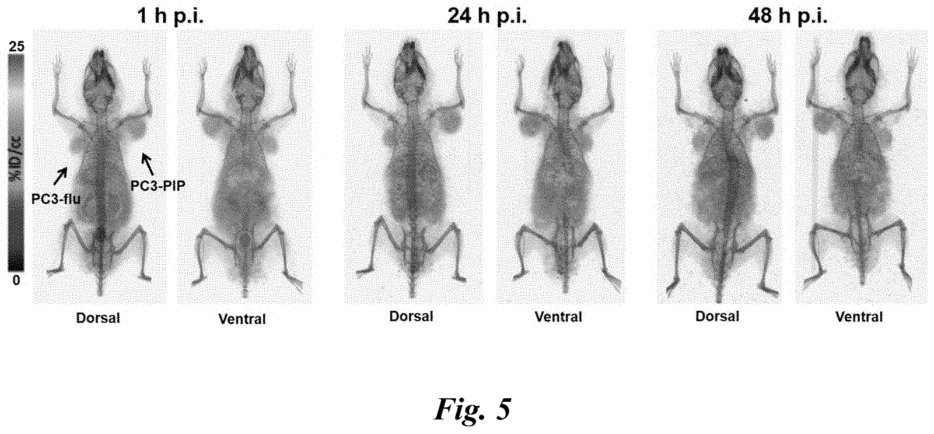

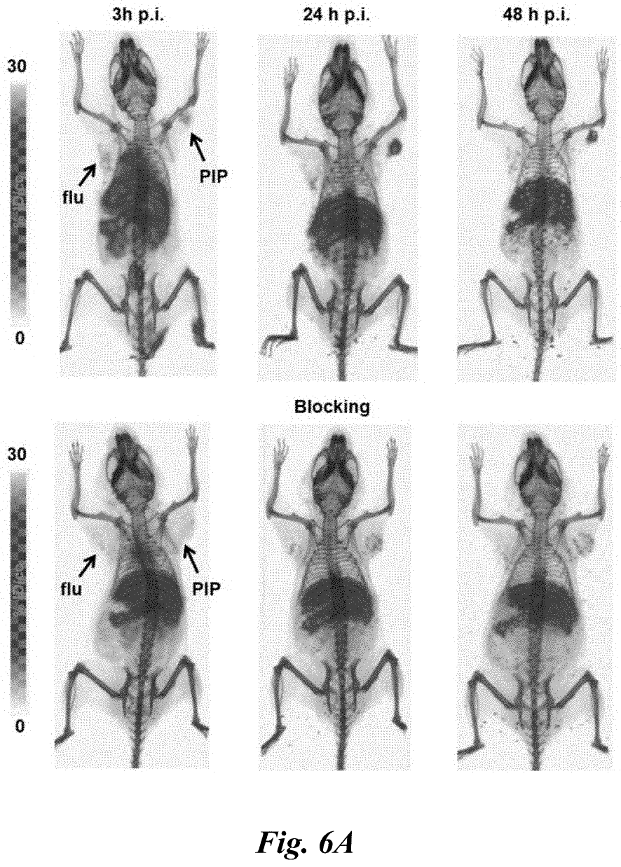

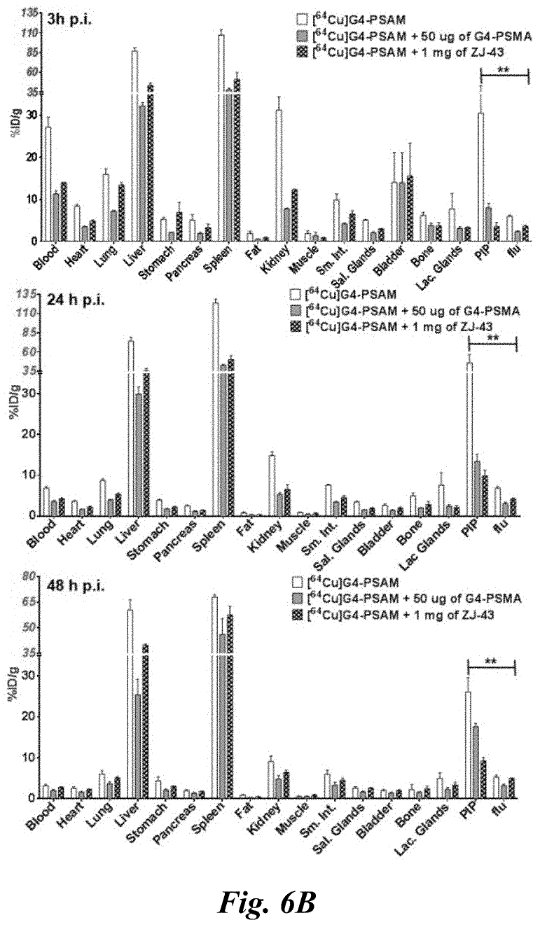

NOD-SCID mice bearing PSMA⁺ PC3 PIP and PSMA⁻ PC3 flu tumors in opposite flanks. In the preliminary studies, one mouse was injected with approximately 200 μCi of [⁶⁴Cu]G4-PSMA and imaged at 1 h, 24 h and 48 h post-injection (FIG. 5) PET-CT imaging acquired at 1 h post-injection (p.i.) shows high background with the highest radioactivity accumulation in bladder and kidneys, followed by liver, spleen, lungs, heart, with modest PSMA⁺ PC3 PIP tumor uptake, which increased at later time points. To avoid intense signal in kidneys and bladder, further PET-CT imaging studies with [⁶⁴Cu]G4-PSMA started at 3 h after injection of [⁶⁴Cu]G4-PSMA (FIG. 6A). Consistently, similar biodistribution of radioactivity was observed, except lower kidney accumulation, indicating fast renal clearance of [⁶⁴Cu]G4-PSMA, facilitated by the nanoparticles approximately 5-nm hydrodynamic radius below renal filtration cut-off. Choi et al., 2007. Uptake of [⁶⁴Cu]G4-PSMA in PSMA⁺ PC3 PIP tumors significantly increased by 24 h and remained high at 48 h after injection. Intravenous administration of [⁶⁴Cu]G4-PSMA resulted in high radioactivity uptake in liver and spleen, most likely due to [⁶⁴Cu] trans-chelation to endogenous proteins, such as ceruloplasmin or albumin. Boswell et al., 2004. Co-administration of [⁶⁴Cu]G4-PSMA with 50 mg/kg of ZJ-43 led to inhibition of radioactivity accumulation in PSMA⁺ PC3 PIP tumors and to some extent in kidneys, in particular at 3 h post-injection, further demonstrating in vivo specificity of G4-PSMA nanoparticles. Kinoshita et al., 2006; Banerjee et al., 2014.

Ex vivo biodistribution of [⁶⁴Cu]G4-PSMA. To validate the PET-CT imaging results, [⁶⁴Cu]G4-PSMA was further evaluated in ex vivo biodistribution studies using the same isogenic PSMA⁺ PC3 PIP and PSMA⁻ PC3 flu tumor models (n=4 or 3). FIG. 6B shows percent of injected dose per gram of tissue in both tumor models, blood and selected organs in three different cohorts injected with [⁶⁴Cu]G4-PSMA (I) or [⁶⁴Cu]G4-PSMA plus unlabeled G4-PSMA (II) or [⁶⁴Cu]G4-PSMA plus ZJ43 (III). Results obtained for cohort I show consistently high accumulation of [⁶⁴Cu]G4-PSMA in PSMA⁺ PC3 PIP tumors with % D/g of 30.56±22.41 at 3 h, 45.83±20.09 at 24 h and 20.41±5.68 at 48 h post-injection. In PSMA⁻ PC3 flu tumors % D/g values were lower: 5.99±0.58, 6.83±1.00 and 5.87±0.94 at the same time points, providing PSMA⁺/PSMA⁻ tumor rations of 4.22±3.74, 7.65±3.35 and 3.94±1.09. Due to relatively long circulation of [⁶⁴Cu]G4-PSMA the PSMA⁺ PC3 PIP/blood ratio was 1.01±0.90 at 3 h p.i., which increased to 6.68±2.93 at 24 h and 5.81±1.62 at 48 h p.i., when blood pool concentration of [⁶⁴Cu]G4-PSMA declined to 6.85±0.85 and 3.51±0.8% D/g, respectively. PSMA⁺ PC3 PIP/muscle ratios were high at all time points. Consistent with renal clearance of [⁶⁴Cu]G4-PSMA and PSMA expression in mouse proximal renal tubules, high accumulation of radioactivity was detected in kidneys and bladder at 3 h p.i., which significantly decreased at 24 h and 48 h p.i. In agreement with PET-CT imaging high radioactivity retention also was detected in liver and spleen. Co-administration of [⁶⁴Cu]G4-PSMA with unlabeled G4-PSMA resulted in radioactivity decline in all analyzed samples, particularly in blood, liver, spleen, kidneys, lacrimal glands and tumors, indicating that biodistribution of [⁶⁴Cu]G4-PSMA strongly depends on its specific activity. PSMA⁺ PC3 PIP/PSMA⁻ PC3 flu, PSMA⁺ PC3 PIP/blood and PSMA⁺ PC3/muscle ratios, however, remained comparable with these derived for mice injected with [⁶⁴Cu]G4-PSMA only, suggesting that the radiotracer maintained its PSMA specificity in spite of low specific activity. Co-administration of [⁶⁴Cu]G4-PSMA and ZJ-43 induced comparable effects to G4-PSMA except considerably lower radioactivity retention in PSMA⁺ PC3 PIP tumors, in particular at 3 h p.i. with PSMA⁺/PSMA⁻ tumor ratios of 1±0.46, 2.71±0.79 and 1.88±0.28 at 3 h, 24 h and 48 h p.i, further verifying PSMA-mediated accumulation of [⁶⁴Cu]G4-PSMA in PSMA⁺ PC3 PIP xenografts.

The discrepancies in biodistribution of G4-PSMA detected by optical imaging and its radioactive counterpart, [⁶⁴Cu]G4-PSMA, observed in PET-CT imaging and ex vivo biodistribution analysis, mainly depicted as high uptake of radioactivity by the liver and spleen, may be attributed to the transchelation of [⁶⁴Cu] to endogenous protein, frequently observed for [⁶⁴Cu]DOTA chelates. Boswell et al., 2004.

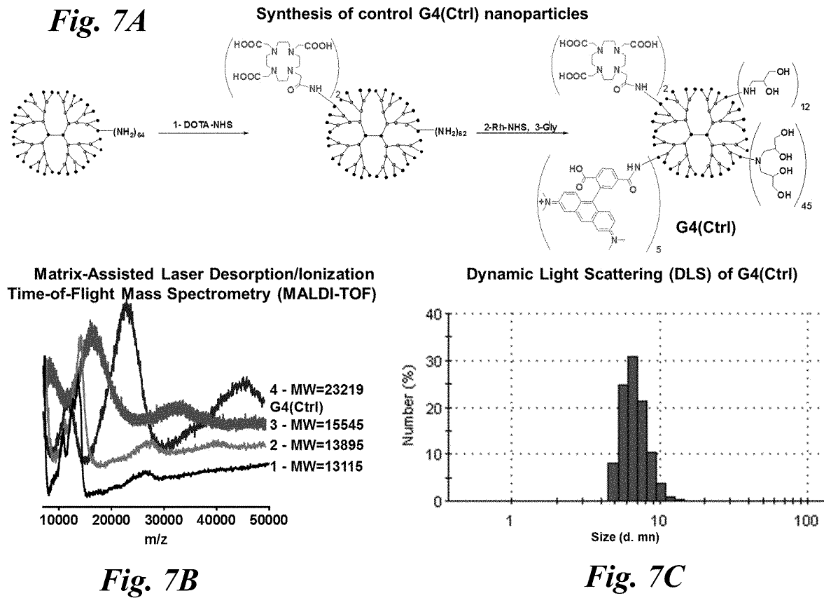

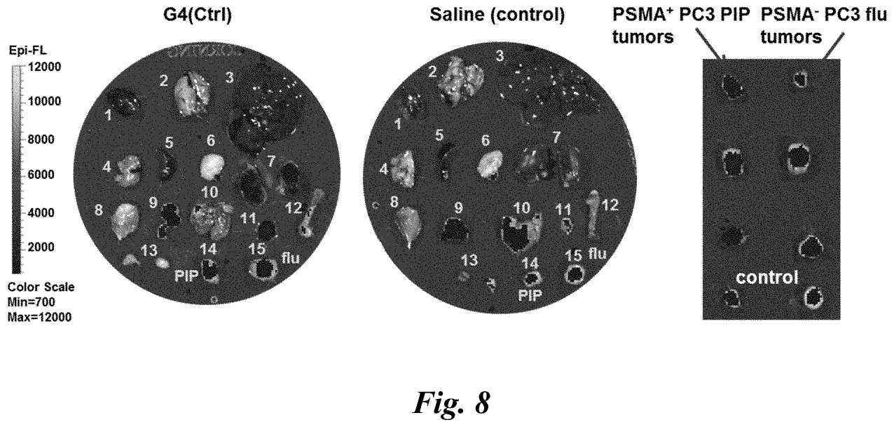

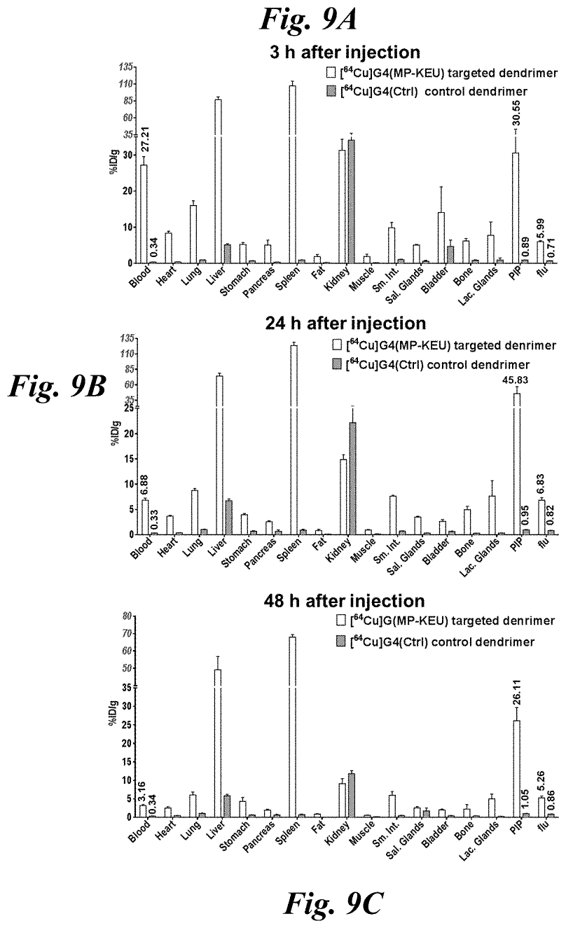

Using the same approach as for G4-PSMA, G4-Ctrl control nanoparticles also were synthesized by conjugating generation 4 amine terminated dendrimer with on average two DOTA chelators and five molecules of rhodamine and capping remaining amines with one hundred two (102) butane-1,2-diol functionalities (FIG. 7). The number of conjugated functionalities was calculated based on an increase of the molecular weight detected upon each synthetic step (FIG. 7B). The resulting nanoparticles exhibited a narrow size distribution as determined by DLS (FIG. 7C). On the contrary to the G4-PSMA targeted dendrimer, G4-Ctrl exhibited low (around 1% ID/g) uptake in both PSMA⁺ PC3 PIP and PSMA⁻ PC3 flu tumors and fast clearance from blood via kidney filtration with significantly lower liver and spleen accumulation (FIG. 8 and FIG. 9).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Ghosh, A.; Heston, W. D. W. Tumor target prostate specific membrane antigen (PSMA) and its regulation in prostate cancer. J Cell Biochem 2004, 91, (3), 528-539.
Chang, S. S.; Reuter, V. E.; Heston, W. D. W.; Gaudin, P. B. Comparison of anti-prostate-specific membrane antigen antibodies and other immunomarkers in metastatic prostate carcinoma. Urology 2001, 57, (6), 1179-1183.
Wright, G. L.; Grob, B. M.; Haley, C.; Grossman, K.; Newhall, K.; Petrylak, D.; Troyer, J.; Konchuba, A.; Schellhammer, P. F.; Moriarty, R. Upregulation of prostate-specific membrane antigen after androgen-deprivation therapy. Urology 1996, 48, (2), 326-334.
Perner, S.; Hofer, M. D.; Kim, R.; Shah, R. B.; Li, H.; Moller, P.; Hautmann, R. E.; Gschwend, J. E.; Kuefer, R.; Rubin, M. A. Prostate-specific membrane antigen expression as a predictor of prostate cancer progression. Human pathology 2007, 38, (5), 696-701.
Mhawech-Fauceglia, P.; Zhang, S.; Terracciano, L.; Sauter, G.; Chadhuri, A.; Herrmann, F. R.; Penetrante, R. Prostate-specific membrane antigen (PSMA) protein expression in normal and neoplastic tissues and its sensitivity and specificity in prostate adenocarcinoma: an immunohistochemical study using mutiple tumour tissue microarray technique. Histopathology 2007, 50, (4), 472-483.
Wang, H. L.; Wang, S. S.; Song, W. H.; Pan, Y.; Yu, H. P.; Si, T. G.; Liu, Y.; Cui, X. N.; Guo, Z. Expression of Prostate-Specific Membrane Antigen in Lung Cancer Cells and Tumor Neovasculature Endothelial Cells and Its Clinical Significance. Plos One 2015, 10, (5).
Haffner, M. C.; Kronberger, I. E.; Ross, J. S.; Sheehan, C. E.; Zitt, M.; Muhlmann, G.; Ofner, D.; Zelger, B.; Ensinger, C.; Yang, X. M. J.; Geley, S.; Margreiter, R.; Bander, N. H. Prostate-specific membrane antigen expression in the neovasculature of gastric and colorectal cancers. Human pathology 2009, 40, (12), 1754-1761.
Baccala, A.; Sercia, L.; Li, J. B.; Heston, W.; Zhou, M. Expression of prostate-specific membrane antigen in tumor-associated neovasculature of renal neoplasms. Urology 2007, 70, (2), 385-390.
Fragomeni, R. A. S.; Menke, J. R.; Holdhoff, M.; Ferrigno, C.; Laterra, J. J.; Solnes, L. B.; Javadi, M. S.; Szabo, Z.; Pomper, M. G.; Rowe, S. P. Prostate-Specific Membrane Antigen-Targeted Imaging With [F-18] DCFPyL in High-Grade Gliomas. Clin Nucl Med 2017, 42, (10), E433-E435.
Kiess, A. P.; Banerjee, S. R.; Mease, R. C.; Rowe, S. P.; Rao, A.; Foss, C. A.; Chen, Y.; Yang, X.; Cho, S. Y.; Nimmagadda, S.; Pomper, M. G. Prostate-specific membrane antigen as a target for cancer imaging and therapy. Q J Nucl Med Mol Im 2015, 59, (3), 241-268.
Maurer, T.; Eiber, M.; Schwaiger, M.; Gschwend, J. E. Current use of PSMA—PET in prostate cancer management. Nat Rev Urol 2016, 13, (4), 226-235.
Zhou, J.; Neale, J. H.; Pomper, M. G.; Kozikowski, A. P. NAAG peptidase inhibitors and their potential for diagnosis and therapy. Nat Rev Drug Discov 2005, 4, (12), 1015-1026.
Kozikowski, A. P.; Nan, F.; Conti, P.; Zhang, J. H.; Ramadan, E.; Bzdega, T.; Wroblewska, B.; Neale, J. H.; Pshenichkin, S.; Wroblewski, J. T. Design of remarkably simple, yet potent urea-based inhibitors of glutamate carboxypeptidase II (NAALADase). Journal of medicinal chemistry 2001, 44, (3), 298-301.
Pomper, M. G.; Musachio, J. L.; Zhang, J. Z.; Scheffel, U.; Zhou, Y.; Hilton, J.; Maini, A.; Dannals, R. F.; Wong, D. F.; Kozikowski, A. P. C-11-MCG: Synthesis, Uptake Selectivity, and Primate PET of a Probe for Glutamate Carboxypeptidase II (NAALADase). Mol Imaging 2002, 1, (2), 96-101.
Burger, I.; Kranzbuehler, B.; Nagel, H.; Huellner, M.; Stolzmann, P.; Eberli, D. PET/MR further improves detection of Ga-68-PSMA imaging for early biochemical recurrence in prostate cancer. J Nucl Med 2017, 58.
Rowe, S. P.; Gorin, M. A.; Hammers, H. J.; Javadi, M. S.; Hawasli, H.; Szabo, Z.; Cho, S. Y.; Pomper, M. G.; Allaf, M. E. Imaging of metastatic clear cell renal cell carcinoma with PSMA-targeted F-18-DCFPyL PET/CT. Ann Nucl Med 2015, 29, (10), 877-882.
Sheikhbahaei, S.; Afshar-Oromieh, A.; Eiber, M.; Solnes, L. B.; Javadi, M. S.; Ross, A. E.; Pienta, K. J.; Allaf, M. E.; Haberkorn, U.; Pomper, M. G.; Gorin, M. A.; Rowe, S. P. Pearls and pitfalls in clinical interpretation of prostate-specific membrane antigen (PSMA)-targeted PET imaging. Eur J Nucl Med Mol I 2017, 44, (12), 2117-2136.
Delker, A.; Fendler, W. P.; Kratochwil, C.; Brunegraf, A.; Gosewisch, A.; Gildehaus, F. J.; Tritschler, S.; Stief, C. G.; Kopka, K.; Haberkorn, U.; Bartenstein, P.; Boning, G. Dosimetry for Lu-177-DKFZ-PSMA-617. a new radiopharmaceutical for the treatment of metastatic prostate cancer. Eur J Nucl Med Mol I2016, 43, (1), 42-51.
Rahbar, K.; Ahmadzadehfar, H.; Kratochwil, C.; Haberkorn, U.; Schafers, M.; Essler, M.; Baum, R. P.; Kulkarni, H. R.; Schmidt, M.; Drzezga, A.; Bartenstein, P.; Pfestroff, A.; Luster, M.; Lutzen, U.; Marx, M.; Prasad, V.; Brenner, W.; Heinzel, A.; Mottaghy, F. M.; Ruf, J.; Meyer, P. T.; Heuschkel, M.; Eveslage, M.; Bogemann, M.; Fendler, W. P.; Krause, B. J. German Multicenter Study Investigating Lu-177-PSMA-617 Radioligand Therapy in Advanced Prostate Cancer Patients. J Nucl Med 2017, 58, (1), 85-90.
Elgamal, A. A. A.; Troychak, M. J.; Murphy, G. P. ProstaScint® scan may enhance identification of prostate cancer recurrences after prostatectomy, radiation, or hormone therapy: Analysis of 136 scans of 100 patients. Prostate 1998, 37, (4), 261-269.
Pandit-Taskar, N.; O'Donoghue, J. A.; Divgi, C. R.; Wills, E. A.; Schwartz, L.; Gonen, M.; Smith-Jones, P.; Bander, N. H.; Scher, H. I.; Larson, S. M.; Morris, M. J. Indium 111-labeled J591 anti-PSMA antibody for vascular targeted imaging in progressive solid tumors. Ejnmmi Res 2015, 5.
Chatalic, K. L. S.; Veldhoven-Zweistra, J.; Bolkestein, M.; Hoeben, S.; Koning, G. A.; Boerman, O. C.; de Jong, M.; van Weerden, W. M. A Novel In-111-Labeled Anti-Prostate-Specific Membrane Antigen Nanobody for Targeted SPECT/CT Imaging of Prostate Cancer. J Nucl Med 2015, 56, (7), 1094-1099.
Chen, Y.; Dhara, S.; Banerjee, S. R.; Byun, Y.; Pullambhatla, M.; Mease, R. C.; Pomper, M. G. A low molecular weight PSMA-based fluorescent imaging agent for cancer. Biochem Bioph Res Co 2009, 390, (3), 624-629.
Chen, Y.; Chatterjee, S.; Lisok, A.; Minn, I.; Pullambhatla, M.; Wharram, B.; Wang, Y. C.; Jin, J. F.; Bhujwalla, Z. M.; Nimmagadda, S.; Mease, R. C.; Pomper, M. G. A PSMA-targeted theranostic agent for photodynamic therapy. J Photoch Photobio B 2017, 167, 111-116.
Ray, S.; Song, X. O. L.; Yang, X.; Minn, I. L.; Lisok, A.; Chatterjee, S.; Chen, J.; van Zijl, P.; McMahon, M.; Pomper, M. Salicylic Acid-Based Polymeric Agents for Targeted Molecular Magnetic Resonance Imaging (MRI) of Prostate Cancer. J Nucl Med 2017, 58.
Liu, G. S.; Banerjee, S. R.; Yang, X.; Yadav, N.; Lisok, A.; Jablonska, A.; Xu, J. D.; Li, Y. G.; Pomper, M. G.; van Zijl, P. A dextran-based probe for the targeted magnetic resonance imaging of tumours expressing prostate-specific membrane antigen. Nat Biomed Eng 2017, 1, (12), 977-+.
Tavakoli, B.; Chen, Y.; Guo, X. Y.; Kang, H. J.; Pomper, M.; Boctor, E. M. Multispectral photoacoustic decomposition with localized regularization for detecting targeted contrast agent. Proc Spie 2015, 9323.
Wang, L. F.; Li, L.; Guo, Y. L.; Tong, H. P.; Fan, X. Z.; Ding, J.; Huang, H. Y. Construction and In Vitro/In Vivo Targeting of PSMA-Targeted Nanoscale Microbubbles in Prostate Cancer. Prostate 2013, 73, (11), 1147-1158.
Petrylak, D. P.; Smith, D. C.; Appleman, L. J.; Fleming, M. T.; Hussain, A.; Dreicer, R.; Sartor, A. O.; Shore, N. D.; Vogelzang, N. J.; Youssoufian, H.; DiPippo, V. A.; Stambler, N.; Huang, K.; Israel, R. J. A phase 2 trial of prostate-specific membrane antigen antibody drug conjugate (PSMA ADC) in taxane-refractory metastatic castration-resistant prostate cancer (mCRPC). J Clin Oncol 2014, 32, (15).
Galsky, M. D.; Eisenberger, M.; Moore-Cooper, S.; Kelly, W. K.; Slovin, S. F.; Delacruz, A.; Lee, Y.; Webb, I J.; Scher, H. I. Phase I trial of the prostate-specific membrane antigen directed immunoconjugate MLN2704 in patients with progressive metastatic castration-resistant prostate cancer. J Clin Oncol 2008, 26, (13), 2147-2154.
Hrkach, J.; Von Hoff, D.; Ali, M. M.; Andrianova, E.; Auer, J.; Campbell, T.; De Witt, D.; Figa, M.; Figueiredo, M.; Horhota, A.; Low, S.; McDonnell, K.; Peeke, E.; Retnarajan, B.; Sabnis, A.; Schnipper, E.; Song, J. J.; Song, Y. H.; Summa, J.; Tompsett, D.; Troiano, G.; Hoven, T. V.; Wright, J.; LoRusso, P.; Kantoff, P. W.; Bander, N. H.; Sweeney, C.; Farokhzad, O. C.; Langer, R.; Zale, S. Preclinical Development and Clinical Translation of a PSMA-Targeted Docetaxel Nanoparticle with a Differentiated Pharmacological Profile. Sci Transl Med 2012, 4, (128).
Farokhzad, O. C.; Cheng, J. J.; Teply, B. A.; Sherifi, I.; Jon, S.; Kantoff, P. W.; Richie, J. P.; Langer, R. Targeted nanoparticle-aptamer bioconjugates for cancer chemotherapy in vivo. P Natl Acad Sci USA 2006, 103, (16), 6315-6320.
Azad, B. B.; Banerjee, S. R.; Pullambhatla, M.; Lacerda, S.; Foss, C. A.; Wang, Y. C.; Ivkov, R.; Pomper, M. G. Evaluation of a PSMA-targeted BNF nanoparticle construct. Nanoscale 2015, 7, (10), 4432-4442.
Zhu, C.; Bandekar, A.; Sempkowski, M.; Banerjee, S. R.; Pomper, M. G.; Bruchertseifer, F.; Morgenstern, A.; Sofou, S. Nanoconjugation of PSMA-Targeting Ligands Enhances Perinuclear Localization and Improves Efficacy of Delivered Alpha-Particle Emitters against Tumor Endothelial Analogues. Molecular cancer therapeutics 2016, 15, (1), 106-113.
Bhatnagar, A.; Wang, Y.; Mease, R. C.; Gabrielson, M.; Sysa, P.; Minn, I.; Green, G.; Simmons, B.; Gabrielson, K.; Sarkar, S.; Fisher, P. B.; Pomper, M. G. AEG-1 promoter-mediated imaging of prostate cancer. Cancer research 2014, 74, (20), 5772-81.
Tse, B. W. C.; Cowin, G. J.; Soekmadji, C.; Jovanovic, L.; Vasireddy, R. S.; Ling, M. T.; Khatri, A.; Liu, T. Q.; Thierry, B.; Russell, P. J. PSMA-targeting iron oxide magnetic nanoparticles enhance MRI of preclinical prostate cancer. Nanomedicine-Uk 2015, 10, (3), 375-386.

Thomas, T. P.; Patri, A. K.; Myc, A.; Myaing, M. T.; Ye, J. Y.; Norris, T. B.; Baker, J. R., Jr. In vitro targeting of synthesized antibody-conjugated dendrimer nanoparticles. Biomacromolecules 2004, 5, (6), 2269-74.
Patri, A. K.; Myc, A.; Beals, J.; Thomas, T. P.; Bander, N. H.; Baker, J. R. Synthesis and in vitro testing of J591 antibody-dendrimer conjugates for targeted prostate cancer therapy. Bioconjugate Chem 2004, 15, (6), 1174-1181.
Huang, B. H.; Otis, J.; Joice, M.; Kotlyar, A.; Thomas, T. P. PSMA-Targeted Stably Linked “Dendrimer-Glutamate Urea-Methotrexate” as a Prostate Cancer Therapeutic. Biomacromolecules 2014, 15, (3), 915-923.
Majer, P.; Jackson, P. F.; Delahanty, G.; Grella, B. S.; Ko, Y. S.; Li, W.; Liu, Q.; Maclin, K. M.; Polakova, J.; Shaffer, K. A.; Stoermer, D.; Vitharana, D.; Wang, E. Y.; Zakrzewski, A.; Rojas, C.; Slusher, B. S.; Wozniak, K. M.; Burak, E.; Limsakun, T.; Tsukamoto, T. Synthesis and biological evaluation of thiol-based inhibitors of glutamate carboxypeptidase II. discovery of an orally active GCP II inhibitor. Journal of medicinal chemistry 2003, 46, (10), 1989-96.
Maresca, K. P.; Hillier, S. M.; Femia, F. J.; Keith, D.; Barone, C.; Joyal, J. L.; Zimmerman, C. N.; Kozikowski, A. P.; Barrett, J. A.; Eckelman, W. C.; Babich, J. W. A series of halogenated heterodimeric inhibitors of prostate specific membrane antigen (PSMA) as radiolabeled probes for targeting prostate cancer. Journal of medicinal chemistry 2009, 52, (2), 347-57.
Mease, R. C.; Dusich, C. L.; Foss, C. A.; Ravert, H. T.; Dannals, R. F.; Seidel, J.; Prideaux, A.; Fox, J. J.; Sgouros, G.; Kozikowski, A. P.; Pomper, M. G. N—[N—[(S)-1,3-dicarboxypropyl]carbamoyl]-4-[F-18]fluorobenzyl-L-cysteine, [F-18]DCFBC: A new imaging probe for prostate cancer. Clin Cancer Res 2008, 14, (10), 3036-3043.
Duncan, R.; Izzo, L. Dendrimer biocompatibility and toxicity. Adv Drug Deliver Rev 2005, 57, (15), 2215-2237.
Choi, H. S.; Liu, W.; Misra, P.; Tanaka, E.; Zimmer, J. P.; Itty Ipe, B.; Bawendi, M. G.; Frangioni, J. V. Renal clearance of quantum dots. Nature biotechnology 2007, 25, (10), 1165-70.
Boswell, C. A.; Sun, X.; Niu, W.; Weisman, G. R.; Wong, E. H.; Rheingold, A. L.; Anderson, C. J. Comparative in vivo stability of copper-64-labeled cross-bridged and conventional tetraazamacrocyclic complexes. Journal of medicinal chemistry 2004, 47, (6), 1465-74.
Kinoshita, Y.; Kuratsukuri, K.; Landas, S.; Imaida, K.; Rovito, P. M., Jr.; Wang, C. Y.; Haas, G. P. Expression of prostate-specific membrane antigen in normal and malignant human tissues. World journal of surgery 2006, 30, (4), 628-36.
Banerjee, S. R.; Pullambhatla, M.; Foss, C. A.; Nimmagadda, S.; Ferdani, R.; Anderson, C. J.; Mease, R. C.; Pomper, M. G. (6)(4)Cu-labeled inhibitors of prostate-specific membrane antigen for PET imaging of prostate cancer. Journal of medicinal chemistry 2014, 57, (6), 2657-69.
Zhang H. K.; Chen Y., Kang J. Lisok A.; Minn I.; Pomper M. G.; Boctor E. M. Prostate-specific membrane antigen-targeted photoacoustic imaging of prostate cancer in vivo. J. Biophotonics. 2018 11(9):e201800021.

Jolanta F. Kukowska-Latallo, Kimberly A. Candido,1 Zhengyi Cao, Shraddha S. Nigavekar, Istvan J. Majoros, Thommey P. Thomas, Lajos P. Balogh, Mohamed K. Khan, and James R. Baker, Jr. Nanoparticle Targeting of Anticancer Drug Improves Therapeutic Response in Animal Model of Human Epithelial Cancer. Cancer Res 2005; 65: (12). Jun. 15, 2005.
Dangshe Ma, Christine E. Hopf, Andrew D. Malewicz, Gerald P. Donovan, Peter D. Senter, William F. Goeckeler, Paul J. Maddon, and William C. Olson, Potent Antitumor Activity of an Auristatin-Conjugated, Fully Human Monoclonal Antibody to Prostate-Specific Membrane Antigen. Clin Cancer Res 2006; 12(8) Apr. 15, 2006;
Xiaojie Li, Munenobu Takashima, Eiji Yuba, Atsushi Harada, and Kenji Kono. PEGylated PAMAM dendrimere doxorubicin conjugate-hybridized gold nanorod for combined photothermal-chemotherapy. Biomaterials 2014 35, 6576-6584; Gail D. Lewis Phillips, Guangmin Li, Debra L. Dugger, Lisa M. Crocker, Kathryn L. Parsons, Elaine Mai, Walter A. Blattler, John M. Lambert, Ravi V. J. Chari, Robert J. Lutz, Wai Lee T. Wong, Frederic S. Jacobson, Hartmut Koeppen, Ralph H. Schwall, Sara R. Kenkare-Mitra, Susan D. Spencer, and Mark X. Sliwkowski. Targeting HER2-Positive Breast Cancer with Trastuzumab-DM1, an Antibody-Cytotoxic Drug Conjugate. Cancer Res 2008; 68:9280-9290.
Christine Tse, et al., Enhanced optical breakdown in KB cells labeled with folate-targeted silver-dendrimer composite nanodevices. Nanomedicine: Nanotechnology, Biology and Medicine, 2011 7 (1), Issue 1, 97-106;
Qian Chen, et al. Targeted CT/MR dual mode imaging of tumors using multifunctional dendrimer-entrapped gold nanoparticles. Biomaterials 2013 34(21), 5200-5209 Wojciech G. Lesniak, et al. Synthesis and Characterization of PAMAM Dendrimer-Based Multifunctional Nanodevices for Targeting αvβ3 Integrins. Bioconjugate Chemistry 2007 18 (4), 1148-1154.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A poly(amidoamine) (PAMAM) dendrimer comprising one or more prostate-specific membrane antigen (PSMA) targeting moieties, one or more optical imaging agents (IA), and one or more chelating moieties (Ch), wherein the one or more chelating moieties optionally comprise a metal or a radiometal suitable for radiotherapy and/or radioimaging, wherein the one or more prostate-specific membrane antigen (PSMA) targeting moieties, one or more optical imaging agents, and one or more chelating moieties are operably linked to the PAMAM dendrimer;
or a pharmaceutically acceptable salt thereof.

2. The PAMAM dendrimer of claim 1, wherein the PAMAM dendrimer is a compound of formula (I): 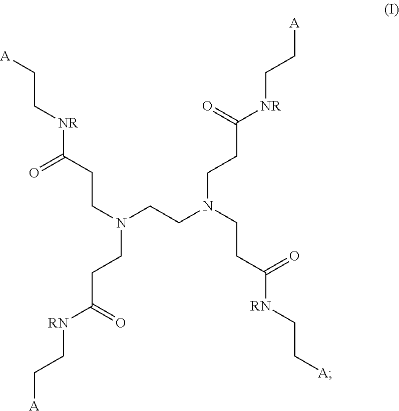
wherein:
each A is: 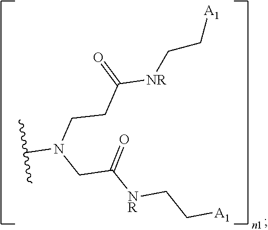
wherein each A₁ is selected from the group consisting of A, a prostate-specific membrane antigen (PSMA) targeting moiety, an optical imaging agent (IA), a chelating moiety (Ch), wherein the chelating moiety optionally comprises a metal or a radiometal suitable for radiotherapy and/or radioimaging, and an end-capping group (EC);
n1 is an integer selected from the group consisting of 1, 2, 3, 4, 5, 6, 7, 8, 9, and 10;
or a pharmaceutically acceptable salt thereof.

3. The PAMAM dendrimer of claim 1 or 2, wherein the PAMAM dendrimer is selected from the group consisting of a generation 0 (G0), a generation 1 (G1), a generation 2 (G2), a generation 3 (G3), a generation 4 (G4), a generation 5 (G5), a generation 6 (G6), a generation 7 (G7), a generation 8 (G8), a generation 9 (G9), and a generation 10 (G10) PAMAM dendrimer.

4. The PAMAM dendrimer of claim 1 or claim 2, wherein the PAMAM dendrimer is a generation four (G4) PAMAM dendrimer.

5. The PAMAM dendrimer of claim 1 or claim 2, wherein the PSMA targeting moiety comprises a Lys-Glu-urea moiety having the following structure: 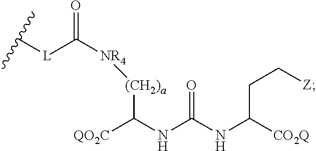
wherein:
Z is tetrazole or CO₂Q;
Q is H or a protecting group;
a is an integer selected from the group consisting of 1, 2, 3, 4, and 5;
R₄ is independently H, substituted or unsubstituted C₁-C₄ alkyl, or —CH₂—R₅;
R₅ is selected from the group consisting of substituted or unsubstituted aryl and substituted or unsubstituted heteroaryl; and
L is a linker.

6. The PAMAM dendrimer of claim 5, wherein the linker (L) is selected from the group consisting of —(CH₂)ₘ₁—, —C(═O)—(CH₂)ₘ₁—, —(CH₂—CH₂—O)ₜ₁—, —C(═O)—(CH₂—CH₂—O)ₜ₁—, —(O—CH₂—CH₂)ₜ₁—, —C(═O)—(O—CH₂—CH₂)ₜ₁—, —C(═O)—(CHR₂)ₘ₁—NR₃—C(═O)—(CH₂)ₘ₁—, —C(═O)—(CH₂)ₘ₁—O—C(═O)—NR₃—(CH₂)ₚ₁—, —C(═O)—(CH₂)ₘ₁—NR₃—C(═O)—O—CH₂)ₚ₁—, —C(═O)—(CH₂)ₘ—NR₃—C(═O)—NR₃—(CH₂)ₚ—, —C(═O)—(CH₂)ₘ—NR₃—C(═O)—(CH₂)ₚ₁—, —C(═O)—(CH₂)ₘ₁—C(═O)—NR₃—(CH₂)ₚ₁—, —C(═O)—(CH₂)ₘ₁—NR₃—C(═O)—NR₃—(CH₂)ₚ₁—, —C(═O)—(CH₂)ₘ₁—NR₁—C(═O)—NR₃—(CH₂)ₚ₁—, —C(═O)—(CH₂)ₘ₁—O—C(═O)—NR₃—, —C(═O)—CH₂)ₘ₁—O—C(═O)—NR₃—(CH₂)ₚ₁—, —C(═O)—(CH₂)ₘ₁—NR₃—C(═O)—O—(CH₂)ₚ₁—, polyethylene glycol, glutaric anhydride, albumin, and one or more amino acids;
wherein:
each R is independently selected from the group consisting of H and C₁-C₄ alkyl;
each R₁ is independently selected from the group consisting of H, Na⁺, C₁-C₄ alkyl, and a protecting group;
each R₂ is independently selected from the group consisting of hydrogen, and —COOR₁;
each R₃ is independently selected from the group consisting of hydrogen, substituted or unsubstituted linear or branched alkyl, alkoxyl, substituted or unsubstituted cycloalkyl, substituted or unsubstituted cycloheteroalkyl, substituted or unsubstituted aryl, substituted or unsubstituted heteroaryl, substituted or unsubstituted arylalkyl, and substituted or unsubstituted heteroarylalkyl;
m1 and p1 are each independently an integer selected from the group consisting of 0, 1, 2, 3, 4, 5, 6, 7 and 8;
t1 is an integer selected from the group consisting of 1, 2, 3, 4, 5, 6, 7, 8, 9, 10, 11 and 12;
wherein the linker is operably bound to the PAMAM dendrimer through a heterobifunctional crosslinker (CL).

7. The PAMAM dendrimer of claim 1 or claim 2, wherein the optical imaging agent (IA) comprises a fluorescent dye.

8. The PAMAM dendrimer of claim 7, wherein the fluorescent dye is selected from the group consisting of rhodamine, rhodamine B, rhodamine 6G, rhodamine 123, carboxytetramethylrhodamine (TAMRA), tetramethylrhodamine (TMR), tetramethylrhodamine-isothiocyanate (TRITC), sulforhodamine 101, Texas Red, Rhodamine Red, Rhodamine Green, AlexaFluor 350, AlexaFluor 405, AlexaFluor 430, AlexaFluor 488, AlexaFluor 500, AlexaFluor 514, AlexaFluor 532, AlexaFluor 546, AlexaFluor 555, AlexaFluor 568, AlexaFluor 594, AlexaFluor 610, AlexaFluor 633, AlexaFluor 635, AlexaFluor 647, BODIPY 630/650, BODIPY 650/665, BODIPY 581/591, BODIPY-FL, BODIPY-R6G, BODIPY-TR, BODIPY-TMR, BODIPY-TRX, Dy677, Dy676, Dy682, Dy752, Dy780, DyLight 350, DyLight 405, DyLight 488, DyLight 547, DyLight 550, DyLight 594, DyLight 633, DyLight 647, DyLight 650, DyLight 680, DyLight 755, DyLight 800, HiLyte Fluor 405, HiLyte Fluor 488, HiLyte Fluor 532, HiLyte Fluor 555, HyLyte Fluor 594, HiLyte Fluor 647, HiLyte Fluor 680, HiLyte Fluor 750, aminomethylcoumarin (AMCA), Cascade Blue, fluorescein, fluorescein isothiocyanate (FITC), Cy3, Cy5, Cy5.5, Cy7, 6-Carboxyfluorescein (6-FAM), and IRDye 800, IRDye 800CW, IRDye 800RS, IRDye 700DX, hexachlorofluorescein (HEX), 6-carboxy-4′,5′-dichloro-2′,7′-dimethoxyfluorescein (6-JOE), Oregon Green 488, Oregon Green 500, Oregon Green 514, Pacific Blue, REG, Renographin, ROX, TET, carbocyanine, indocarbocyanine, oxacarbocyanine, thuicarbocyanine, merocyanine, polymethine, coumarine, xanthene, a boron-dipyrromethane VivoTag-680, VivoTag-S680, VivoTag-S750, dimethyl{4-[1,5,5-tris(4-dimethylaminophenyl)-2,4-pentadienylidene]-2,5-cyclohexadien-1-ylidene}ammonium perchlorate (IR800), ADS780WS, ADS830WS, ADS832WS, R-Phycoerythrin, Flamma749, Flamma774, and indocyanine green (ICG), and N-hydroxysuccinimide (NHS) esters, maleimides, phosphines, and free acids thereof.

9. The PAMAM dendrimer of claim 1 or claim 2, wherein the chelating moiety (Ch) is selected from the group consisting of 1,4,7,10-tetraazacyclododecane-1,4,7,10-tetraacetic acid (DOTA) or a DOTA analog, or any other metal chelator, such as diethylenetriamine pentaacetic acid (DTPA), N′-{5-[Acetyl(hydroxy)amino]pentyl}-N-[5-({4-[(5-aminopentyl)(hydroxy)amino]-4-oxobutanoyl}amino)pentyl]-N-hydroxysuccinamide (DFO), and 1,4,7-triazacyclononane-N,N′,N″-triacetic acid (NOTA).

10. The PAMAM dendrimer of claim 1 or claim 2, wherein the chelating moiety (Ch) is selected from the group consisting of:

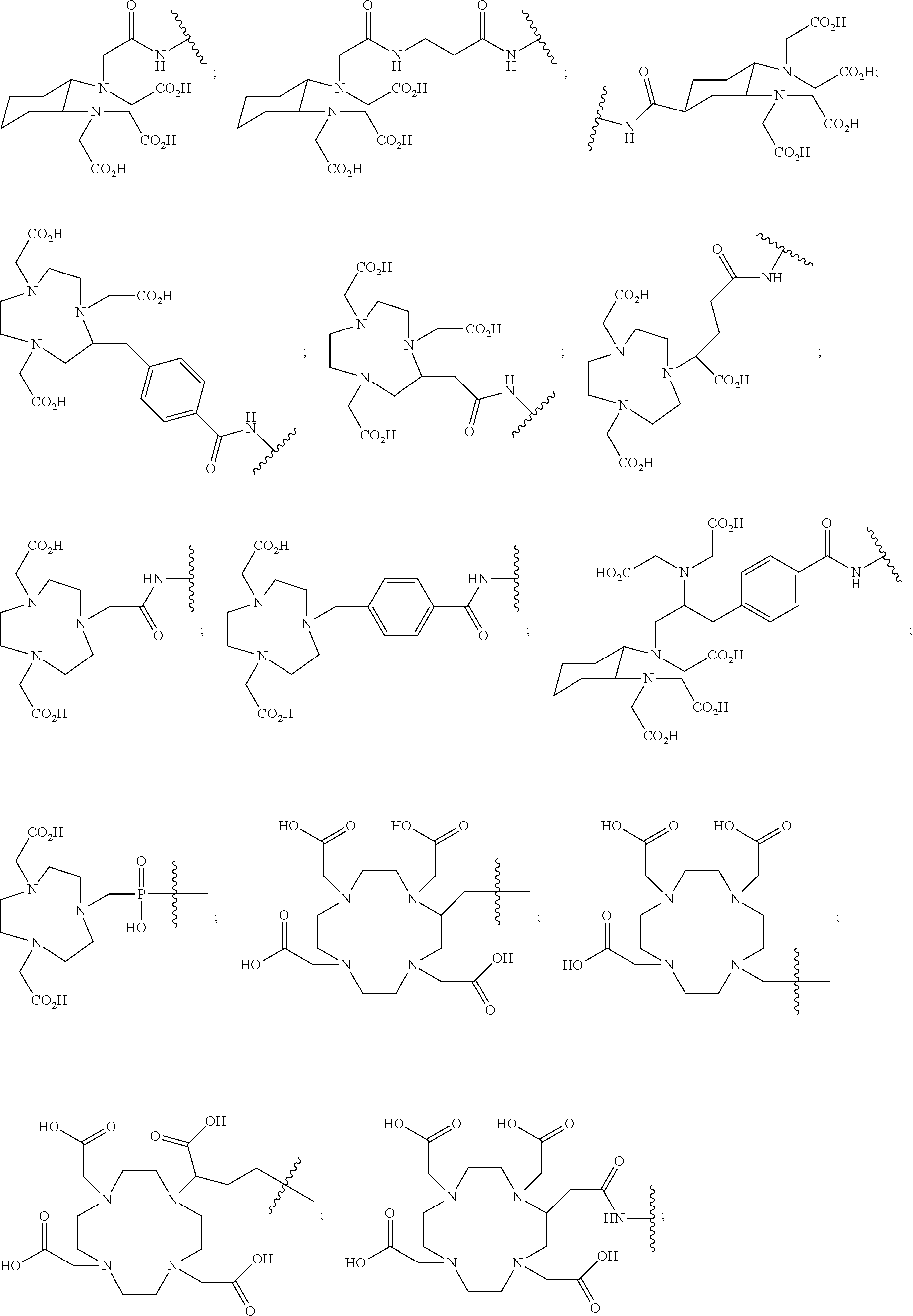

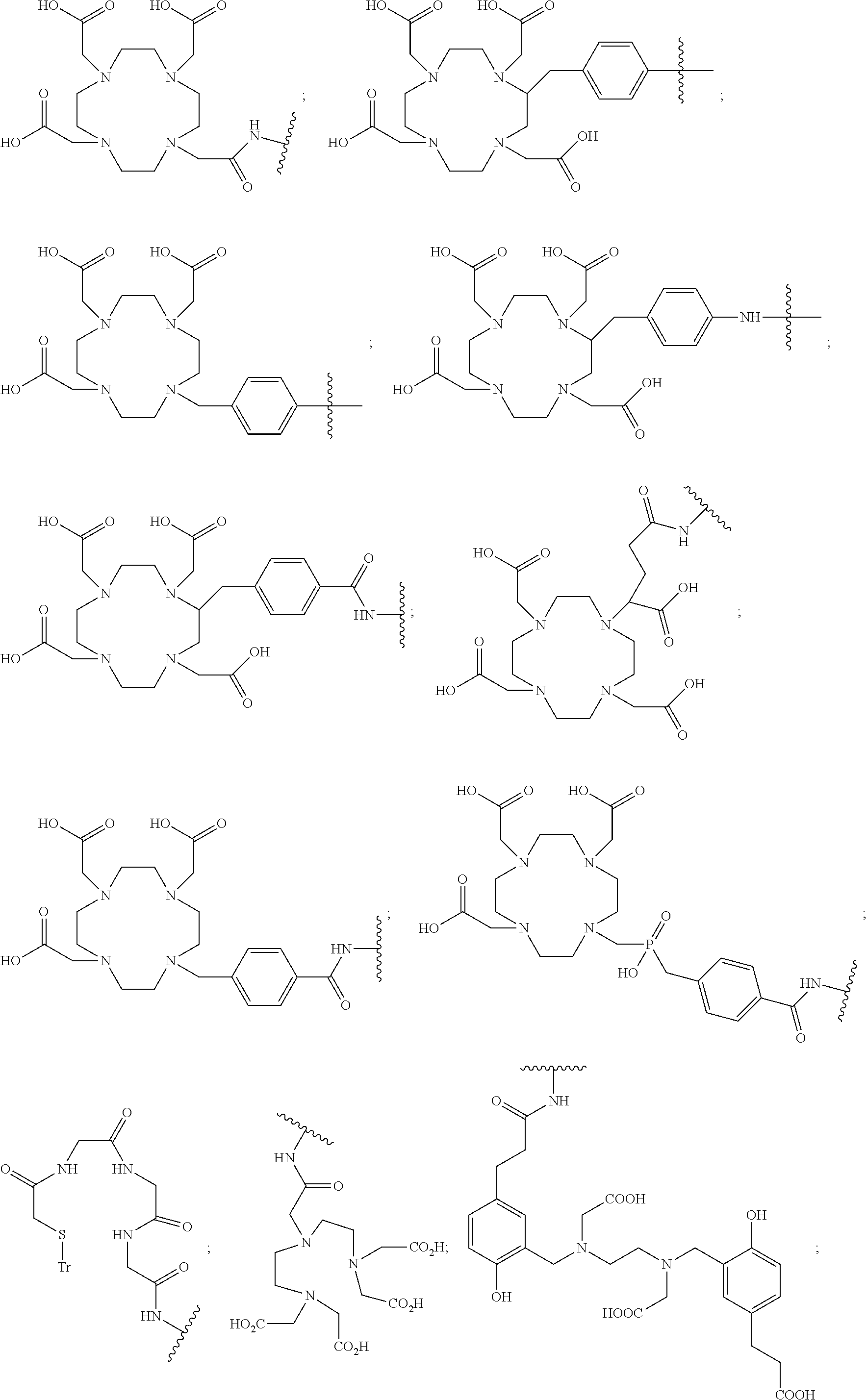

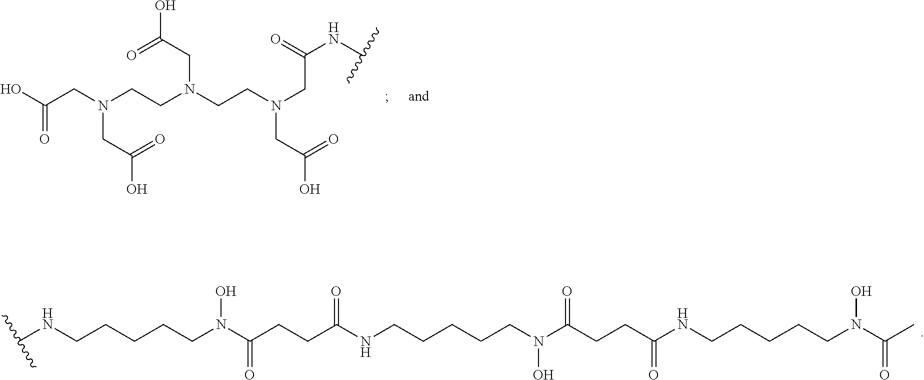 [SMILES: *NC(=O)CN(CCN(CCN(CC(=O)O)CC(=O)O)CC(=O)O)CC(=O)O ; *NCCCCCN(O)C(=O)CCC(=O)NCCCCCN(O)C(=O)CCC(=O)NCCCCCN(O)C(C)=O]

11. The PAMAM dendrimer of claim 10, wherein the chelating moiety is selected from the group consisting of.

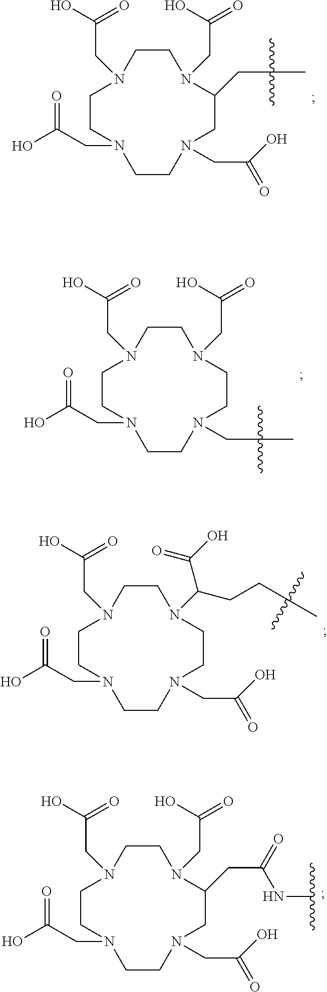 [SMILES: *CC1CN(CC(=O)O)CCN(CC(=O)O)CCN(CC(=O)O)CCN1CC(=O)O ; *CN1CCN(CC(=O)O)CCN(CC(=O)O)CCN(CC(=O)O)CC1 ; *CCC(C(=O)O)N1CCN(CC(=O)O)CCN(CC(=O)O)CCN(CC(=O)O)CC1 ; *NC(=O)CC1CN(CC(=O)O)CCN(CC(=O)O)CCN(CC(=O)O)CCN1CC(=O)O]

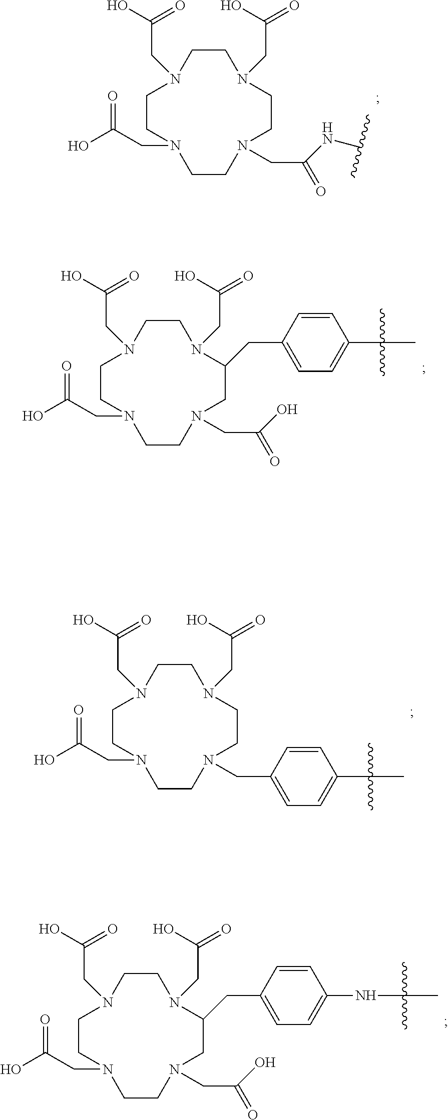 [SMILES: *NC(=O)CN1CCN(CC(=O)O)CCN(CC(=O)O)CCN(CC(=O)O)CC1 ; *c1ccc(CC2CN(CC(=O)O)CCN(CC(=O)O)CCN(CC(=O)O)CCN2CC(=O)O)cc1 ; *c1ccc(CN2CCN(CC(=O)O)CCN(CC(=O)O)CCN(CC(=O)O)CC2)cc1 ; *Nc1ccc(CC2CN(CC(=O)O)CCN(CC(=O)O)CCN(CC(=O)O)CCN2CC(=O)O)cc1]

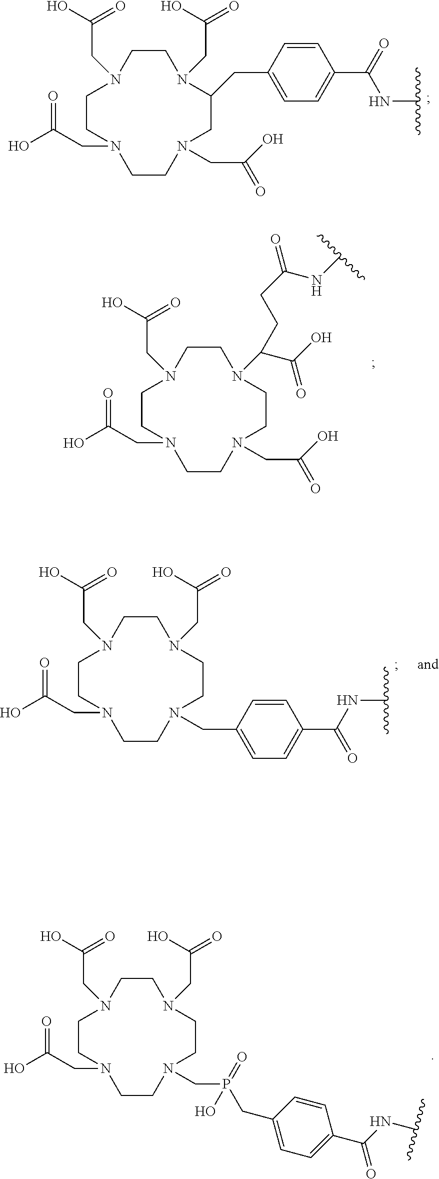 [SMILES: *NC(=O)c1ccc(CC2CN(CC(=O)O)CCN(CC(=O)O)CCN(CC(=O)O)CCN2CC(=O)O)cc1 ; *NC(=O)CCC(C(=O)O)N1CCN(CC(=O)O)CCN(CC(=O)O)CCN(CC(=O)O)CC1 ; *NC(=O)c1ccc(CN2CCN(CC(=O)O)CCN(CC(=O)O)CCN(CC(=O)O)CC2)cc1 ; *NC(=O)c1ccc(CP(=O)(O)CN2CCN(CC(=O)O)CCN(CC(=O)O)CCN(CC(=O)O)CC2)cc1]

12. The PAMAM dendrimer of claim 1 or claim 2, wherein the metal is selected from the group consisting of Cu, Ga, Zr, Y, Tc, In, Lu, Bi, Mn, Ac, Ra, Re, Sm, Al—F, and Sr.

13. The PAMAM dendrimer of claim 12, wherein the metal is a radiometal and the radiometal is selected from the group consisting of ⁶⁴Cu, ⁶⁷Cu, ⁶⁸Ga, ⁶⁰Ga, ⁸⁹Zr, ⁸⁶Y, ⁹⁰Y, ^{94m}Tc, ¹¹¹In, ⁶⁷Ga, ^{99m}Tc, ¹⁷⁷Lu, ⁵²Mn, ²¹³Bi, ²¹²Bi, ⁹⁰Y, ²¹¹At, ²²⁵Ac, ²²³Ra, ^{186/188}Re, ¹⁵³Sm, Al¹⁸F, and ⁸⁹Sr.

14. The PAMAM dendrimer of claim 2, wherein the end-capping group (EC) is selected from the group consisting of —NH₂, —(CH₂)ₘ₁—CH₂—CH(OR₁)—(CH₂)ₘ₁—OR₁, —NR—(CH₂)ₘ₁—CH(OR₁)—(CH₂)ₘ₁—OR₁, —NR—C(═O)—CH₃, —C(═O)—O—Na+, —C(═O)—NR—(CH₂)ₘ₁—OR₁, —NR—C(═O)—(CH₂)ₘ₁—C(═O)OR₁, and —NR—(CH₂)ₘ₁—CH(OR₁)—(CH₂)ₘ₁—CH₃;
wherein:
each R is independently selected from the group consisting of H and C₁-C₄ alkyl;
each R₁ is independently selected from the group consisting of H, Na⁺, C₁-C₄ alkyl, and a protecting group; and
each m1 is independently an integer selected from the group consisting of 0, 1, 2, 3, 4, 5, 6, 7, 8, 9, 10, 11, and 12.

15. The PAMAM dendrimer of claim 1 or claim 2 further comprising a heterobifunctional crosslinker (CL).

16. The PAMAM dendrimer of claim 6 or claim 15, wherein the heterobifunctional crosslinker (CL) is selected from the group consisting of succinimidyl 4-(N-maleimidomethyl)cyclohexane-1-carboxylate (SMCC), m-maleimidobenzoyl-N-hydroxysuccinimide ester (MBS), N-(beta-maleimidopropyloxy)succinimide ester (BMPS), N-[e-maleimidocaproyloxy]succinimide ester (EMCS), N-[gamma-maleimidobutyryloxy]succinimide (GMBS), N-succinimidyl 4-[4-maleimidophenyl]butyrate (SMPB), succinimidyl-6-(β-maleimidopropionamido)hexanoate (SMPH), and maleimide-polyethylene glycol-N-hydroxysuccinimide ester (MAL-PEG-NHS).

17. The PAMAM dendrimer of claim 1 or claim 2, wherein the PAMAM dendrimer has the following chemical structure: [symbol]
wherein:
m, n, p, q, and t are each independently integers from 0 to 64;
Ch is a chelating moiety;
CL is a heterobifunctional crosslinker;
EC is an end-capping group;
IA is an optical imaging agent; and
PSMA is a PSMA-targeting moiety.

18. The PAMAM dendrimer of claim 17, wherein the PAMAM dendrimer has the following chemical structure:

[symbol]

19. A pharmaceutical composition comprising a PAMAM dendrimer of any of claims 1-16, and a pharmaceutically acceptable carrier, diluent, or excipient.

20. A method for imaging or treating one or more PSMA expressing tumors or cells, the method comprising contacting the one or more PSMA expressing tumors or cells with an effective amount of a PAMAM dendrimer of any of claims 1-18, or a pharmaceutical composition thereof.

21. The method of claim 20, wherein the imaging or treating is in vitro, in vivo, or ex vivo.

22. The method of claim 20, wherein the imaging is positron emission tomography (PET) and the radiometal is selected from the group consisting of ⁶⁴Cu, ⁶⁷Cu, ⁶⁸Ga, ⁶⁰Ga, ⁸⁹Zr, ⁸⁶Y, and ^{94m}Tc.

23. The method of claim 20, wherein the imaging is single-photon emission computed tomography (SPECT) and the radiometal is selected from the group consisting of ¹¹¹In, ⁶⁷Ga, ^{99m}Tc, and ¹⁷⁷Lu.

24. The method of claim 20, further comprising diagnosing, based on the image, a disease or condition in a subject.

25. The method of claim 20, further comprising monitoring, based on the image, progression or regression of a disease or condition in a subject.

26. The method of claim 20, wherein the treating comprises radiotherapy.

27. The method of claim 26, wherein the radiotherapy comprises a radiometal suitable for radiotherapy selected from the group consisting of ¹⁷⁷Lu, ²¹³Bi, ²¹²Bi, ⁹⁰Y ²¹¹At, ²²⁵Ac, ²²³R, and ⁸⁹Sr.

28. The method of claim 20, wherein the method comprises imaging or treating a cancer.

29. The method of claim 28, wherein the cancer is selected from the group consisting of a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

30. Use of a PAMAM dendrimer of any of claims 1-18, or a pharmaceutical composition thereof, as a chelating agent for magnetic resonance imaging (MRI); in photodynamic therapy; in photoacoustic imaging; for drug delivery; for encapsulating metallic clusters; for computerized tomography (CT) imaging; and as a nanodevice.
